(19) United States
(12) Patent Application Publication
(10) Pub. No.: US 2026/0109742 A1
(43) Pub. Date: Apr. 23, 2026

(54) RECOMBINANT SPIRULINA EXPRESSING SCAFFOLDS AND METHODS OF UTILIZING THE SAME

(71) Applicant: Lumen Bioscience, Inc., Seattle, WA (US)

(72) Inventors: James Michael ROBERTS, Seattle, WA (US); Nhi Yen KHUONG, Kenmore, WA (US); Mesfin Mulugeta GEWE, Bothell, WA (US); Benjamin JESTER, Seattle, WA (US)

(21) Appl. No.: 19/422,077

(22) Filed: Dec. 16, 2025

Related U.S. Application Data
(63) Continuation of application No. PCT/US2024/036189, filed on Jun. 28, 2024.
(60) Provisional application No. 63/511,050, filed on Jun. 29, 2023.

(51) Int. Cl. C07K 14/47; A61K 38/00; C12N 15/74

(52) U.S. Cl. CPC C07K 14/47; C12N 15/74; A61K 38/00; C07K 2319/30

(57) ABSTRACT

The present disclosure provides scaffolds comprising heterologous moieties and Spirulina expressing the same. Also provided are recombinant Spirulina expressing scaffolds and methods of utilizing the same for the treatment of diseases and conditions.

### CROSS REFERENCE TO RELATED APPLICATIONS

This application is a continuation of International Application No. PCT/US2024/036189, filed Jun. 28, 2024, which claims priority to U.S. Application No. 63/511,050, filed on Jun. 29, 2023, the contents of each of which is incorporated by reference herein in its entirety for all purposes.

### REFERENCE TO AN ELECTRONIC SEQUENCE LISTING

The contents of the electronic sequence listing (LUBI_038_01US_SeqList_ST26.xml; Size: 82,286 bytes; and Date of Creation: Dec. 12, 2025) are herein incorporated by reference in their entirety.

### BACKGROUND

Antigen-binding domains such as VHHs from camelid single-chain antibodies are expressible in prokaryotes like Spirulina. However, various limitations remain such as the requirement for affinity maturation to achieve improved target binding.

The present disclosure provides a resolution to the limitations of the current state-of-the-art via improved compositions comprising scaffolds.

### FIELD

The present disclosure relates to scaffolds and methods of utilizing the same. The disclosure also relates to constructs comprising scaffolds expressing binding agents.

### BRIEF SUMMARY

Provided herein are recombinant Spirulina, that expresses a mutated small-membrane A-kinase anchoring protein (smAKAP) peptide sequence. In aspects, the Spirulina comprises at least 2, 3, 4, 5, 6, or 7 mutated residues in the mutant smAKAP peptide sequence as compared to a WT smAKAP peptide sequence. In aspects, 1 residue in the smAKAP peptide sequence is mutated as compared to a WT smAKAP peptide sequence. In aspects, 2 residues in the smAKAP peptide sequence are mutated as compared to a WT smAKAP peptide sequence. In aspects, 3 residues in the smAKAP peptide sequence are mutated as compared to a WT smAKAP peptide sequence. In aspects, the mutated smAKAP peptide exhibits resistance to protease cleavage as determined by reduced detection of cleavage products when exposed to a solvent comprising a protease for one hour. In aspects, at most about 5%, 10%, 20%, 30%, 40%, 50%, or 60% of the smAKAP peptide sequence is cleaved. In aspects, the mutation is of a hydrophobic residue. In aspects, when the recombinant Spirulina is submerged in a solvent, the mutated residue is exposed to the solvent. In aspects, the mutated residue is selected from the group consisting of: C16S, C24S, E5D, Y6H, W22S, C24G, L4E, R9E, LAI, L10I, L19I, and combination thereof of SEQ ID NO: 2. In aspects, the mutated residue is C16S and C24S as compared to SEQ ID NO: 2. In aspects, the mutated residue is E5D, Y6H, C16S, W22S, and C24G as compared to SEQ ID NO: 2. In aspects, the mutated residue is L4E, E5D, Y6H, R9E, C16S, W22S, and C24G as compared to SEQ ID NO: 2. In aspects, the mutated residue is L4E, C16S, W22S, and C24G as compared to SEQ ID NO: 2. In aspects, the mutated residue is R9E, C16S, and C24G as compared to SEQ ID NO: 2. In aspects, the mutated residue is R9E, C16S, W22S, and C24G as compared to SEQ ID NO: 2. In aspects, the mutant smAKAP peptide sequence is linked to a heterologous moiety in a monomeric configuration. In aspects, the mutant smAKAP peptide sequence is linked to a second heterologous moiety. In aspects, the heterologous moiety and the second heterologous moiety are the same. In aspects, the heterologous moiety and the second heterologous moiety are the different. In aspects, the recombinant Spirulina expresses another exogenous polypeptide sequence. In aspects, the exogenous polypeptide sequence is selected from the group consisting of: oligomerization domain of C4b-binding protein (C4BP), cholera toxin b subunit, oligomerization domains of extracellular matrix proteins, TRX, 5HVZ, cTRP, SP651, SP737, and 4B0F. In aspects, the exogenous polypeptide sequence is 5HVZ. In aspects, the 5HVZ is linked to a third heterologous moiety. In aspects, the third heterologous moiety is in a homodimeric configuration. In aspects, the heterologous moiety, the second heterologous moiety, and the third heterologous moieties are the same. In aspects, the heterologous moiety, the second heterologous moiety, and the third heterologous moieties are the different. In aspects, the heterologous moiety is a binding agent. In aspects, the binding agent is selected from the group consisting of: fab′, F(ab′) 2, fv, domain antibody (dAb), complementarity Determining Region (CDR) fragment, CDR-grafted antibody, single chain antibodies (scFv), single chain antibody fragment, chimeric antibody, diabody, triabody, tetrabody, minibody, linear antibody, intrabody, nanobody (single domain antibody), small Modular Immunopharmaceuticals (SMIPs), antigen-binding domain immunoglobulin fusion protein, and VHH. In aspects, the binding agent is the VHH. In aspects, the VHH binds a pathogen. In aspects, the VHH binds a cancer cell. In aspects, the VHH binds a human cell. In aspects, the VHH binds a pathogen selected from the group consisting of: bacteria, fungi, and virus. In aspects, the pathogen is selected from the group consisting of: E. coli, Enterotoxigenic E. coli (ETEC), anthrax, EHEC, EAEC, Shigella, Mycobacterium, Streptococcus, Staphylococcus, Shigella, Campylobacter, Salmonella, Clostridium, Corynebacterium, Pseudomonas, Neisseria, Listeria, Vibrio, Bordetella, Legionella, bacteriophage, RNA bacteriophage (e.g. MS2, AP205, PP7 and QB), Helicobacter pylori, Infectious Hematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, White Spot Syndrome Virus, a coronavirus, SARS, MERS, SARS-COV-2, Aspergillus, Candida, Blastomyces, Coccidioides, Cryptococcus, Histoplasma, Plasmodium, P. falciparum, P. malariae, P. ovale, P. vivax, Trypanosoma, Toxoplasma, Giardia, Leishmania Cryptosporidium, helminthic parasites: Trichuris spp., Enterobius spp., Ascaris spp., Ancylostoma spp. and Necatro spp., Strongyloides spp., Dracunculus spp., Onchocerca spp. Wuchereria spp., Taenia spp., Echinococcus spp., and Diphyllobothrium spp., Fasciola spp., and Schistosoma spp. In aspects, the pathogen is bacteria and the bacteria is selected from the group consisting of: Mycobacterium, Streptococcus, Staphylococcus, Shigella, Campylobacter, Salmonella, Clostridium, Corynebacterium, Pseudomonas, Neisseria, Listeria, Vibrio, Bordetella, and Legionella. In aspects, the bacteria is Campylobacter. In aspects, the bacteria is Clostridium. In aspects, the VHH comprises a sequence with at least 85% identity to a sequence of SEQ ID NO: 25-67. In aspects, the VHH comprises a sequence SEQ ID NO: 25-67. In aspects, the recombinant Spirulina expresses a scaffold having at least 85%, 90%, 95%, 96%, 97%, 98%, 99%, or 100% identity to a sequence of SEQ ID NO: 6, 14, 16, 18, 20, 22, 23, and 24.

Provided are also recombinant Spirulina, that expresses a mutated small-membrane A-kinase anchoring protein (smAKAP) peptide sequence, wherein the mutation comprises a substitution of a residue of SEQ ID NO: 2.

Provided are also recombinant Spirulina, that expresses a mutated small-membrane A-kinase anchoring protein (smAKAP) peptide sequence, wherein the mutation is selected from the group consisting of: C16S, C24S, E5D, Y6H, W22S, C24G, L4E, R9E, LAI, L10I, L19I, and combination thereof of SEQ ID NO: 2. In aspects, smAKAP is linked to one or more VHH antibodies at a terminal end. In aspects, the smAKAP is linked to two VHH antibodies, wherein a first VHH is at a C-terminus and the second VHH is at an N-terminus. In aspects, the recombinant Spirulina expresses a 5HVZ. In aspects, the 5HVZ is linked to two VHH antibodies in a homodimeric configuration.

Provided are also polynucleotide sequences comprising a mutated small-membrane A-kinase anchoring protein (smAKAP) sequence, wherein the mutation a single residue substitution as compared to a WT smAKAP sequence.

Provided are also vectors comprising polynucleotide sequences.

Provided are also methods of making a recombinant Spirulina comprising contacting a Spirulina cell with a vector of the disclosure.

Provided are also pharmaceutical compositions comprising recombinant Spirulina and an excipient.

Provided are also kits comprising: recombinant Spirulina, polynucleotide sequences, vectors, or pharmaceutical compositions, and instructions for use thereof.

Provided are also methods of treatment comprising administering subject pharmaceutical compositions to a subject in need thereof, wherein the subject comprises a bacterial or viral infection.

Provided are also vectors comprising a nucleotide sequence encoding a polypeptide of SEQ ID NO: 5-7, or 14-24.

Provided are also recombinant Spirulina comprising vectors of the disclosure.

Provided are recombinant Spirulina, that express a mutated small-membrane A-kinase anchoring protein (smAKAP) peptide sequence. In aspects, Spirulina comprise at least 2, 3, 4, 5, 6, or 7 mutated residues in the mutant smAKAP peptide sequence as compared to a WT smAKAP peptide sequence. In aspects, 1 residue in the smAKAP peptide sequence is mutated as compared to a WT smAKAP peptide sequence. In aspects, 2 residues in the smAKAP peptide sequence are mutated as compared to a WT smAKAP peptide sequence. In aspects, 3 residues in the smAKAP peptide sequence are mutated as compared to a WT smAKAP peptide sequence. In aspects, a mutated smAKAP peptide exhibits resistance to protease cleavage as determined by reduced detection of cleavage products when exposed to a solvent comprising a protease for one hour. In aspects, at most about 5%, 10%, 20%, 30%, 40%, 50%, or 60% of the smAKAP peptide sequence is cleaved. In aspects, a mutation is of a hydrophobic residue. In aspects, a recombinant Spirulina is submerged in a solvent, the mutated residue is exposed to the solvent. In aspects, a mutated residue is selected from the group consisting of: C16S, C24S, E5D, Y6H, W22S, C24G, L4E, R9E, LAI, L10I, L19I, and combination thereof of SEQ ID NO: 2. In aspects, a mutated residue is C16S and C24S as compared to SEQ ID NO: 2. In aspects, a mutated residue is E5D, Y6H, C16S, W22S, and C24G as compared to SEQ ID NO: 2. In aspects, a mutated residue is L4E, E5D, Y6H, R9E, C16S, W22S, and C24G as compared to SEQ ID NO: 2. In aspects, a mutated residue is L4E, C16S, W22S, and C24G as compared to SEQ ID NO: 2. In aspects, a mutated residue is R9E, C16S, and C24G as compared to SEQ ID NO: 2. In aspects, a mutated residue is R9E, C16S, W22S, and C24G as compared to SEQ ID NO: 2. In aspects, a mutated residue is L4I, R9E, C16S, C24G as compared to SEQ ID NO: 2. In aspects, a mutant smAKAP peptide sequence is linked to a heterologous moiety in a monomeric configuration. In aspects, a mutant smAKAP peptide sequence is linked to a second heterologous moiety. In aspects, a heterologous moiety and the second heterologous moiety are the same. In aspects, a heterologous moiety and the second heterologous moiety are the different. In aspects, a recombinant Spirulina expresses another exogenous polypeptide sequence. In aspects, an exogenous polypeptide sequence is selected from the group consisting of: oligomerization domain of C4b-binding protein (C4BP), cholera toxin b subunit, oligomerization domains of extracellular matrix proteins, TRX, 5HVZ, cTRP, SP651, SP737, and 4B0F. In aspects, an exogenous polypeptide sequence is 5HVZ. In aspects, a 5HVZ is linked to a third heterologous moiety. In aspects, a third heterologous moiety is in a dimeric configuration. In aspects, a heterologous moiety, the second heterologous moiety, and the third heterologous moieties are the same. In aspects, a heterologous moiety, the second heterologous moiety, and the third heterologous moieties are the different. In aspects, a heterologous moiety is a binding agent. In aspects, a binding agent is selected from the group consisting of: fab′, F (ab′) 2, fv, domain antibody (dAb), complementarity Determining Region (CDR) fragment, CDR-grafted antibody, single chain antibodies (scFv), single chain antibody fragment, chimeric antibody, diabody, triabody, tetrabody, minibody, linear antibody, intrabody, nanobody (single domain antibody), small Modular Immunopharmaceuticals (SMIPs), antigen-binding domain immunoglobulin fusion protein, and VHH. In aspects, a binding agent is a VHH. In aspects, a VHH binds a pathogen. In aspects, a VHH binds a cancer cell. In aspects, a VHH binds a human cell. In aspects, a VHH binds a pathogen selected from the group consisting of: bacteria, fungi, and virus. In aspects, a pathogen is selected from the group consisting of: E. coli, Enterotoxigenic E. coli (ETEC), anthrax, EHEC, EAEC, Shigella, Mycobacterium, Streptococcus, Staphylococcus, Shigella, Campylobacter, Salmonella, Clostridium, Corynebacterium, Pseudomonas, Neisseria, Listeria, Vibrio, Bordetella. Legionella, bacteriophage, RNA bacteriophage (e.g. MS2, AP205, PP7 and QB), Helicobacter pylori, Infectious Hematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, White Spot Syndrome Virus, a coronavirus, SARS, MERS, SARS-CoV-2, Aspergillus, Candida, Blastomyces, Coccidioides, Cryptococcus, Histoplasma, Plasmodium, P. falciparum, P. malariae, P. ovale, P. vivax, Trypanosoma, Toxoplasma, Giardia, Leishmania Cryptosporidium, helminthic parasites: Trichuris spp., Enterobius spp., Ascaris spp., Ancylostoma spp. and Necatro spp., Strongyloides spp., Dracunculus spp., Onchocerca spp. Wuchereria spp., Taenia spp., Echinococcus spp., and Diphyllobothrium spp., Fasciola spp., and Schistosoma spp. In aspects, a pathogen is bacteria and the bacteria is selected from the group consisting of: Mycobacterium, Streptococcus, Staphylococcus, Shigella, Campylobacter, Salmonella, Clostridium, Corynebacterium, Pseudomonas, Neisseria, Listeria, Vibrio, Bordetella, and Legionella. In aspects, a bacterium is Campylobacter. In aspects, a bacterium is Clostridium. In aspects, a VHH comprises a sequence with at least 85% identity to a sequence of SEQ ID NO: 25-67. In aspects, a VHH comprises a sequence of SEQ ID NO: 25-67. In aspects, recombinant Spirulina express a scaffold having at least 85%, 90%, 95%, 96%, 97%, 98%, 99%, or 100% identity to a sequence of SEQ ID NO: 4, 6, 14, 16, 18, 20, 22, 23, 24, and 76.

Provided are recombinant Spirulina, that express a mutated small-membrane A-kinase anchoring protein (smAKAP) peptide sequence, wherein the mutation comprises a substitution of a residue of SEQ ID NO: 2.

Provided are recombinant Spirulina, that express a mutated small-membrane A-kinase anchoring protein (smAKAP) peptide sequence, wherein the mutation is selected from the group consisting of: C16S, C24S, E5D, Y6H, W22S, C24G, L4E, R9E, L4I, L10I, L19I, and combination thereof of SEQ ID NO: 2. In aspects, the smAKAP is linked to one or more VHH antibodies at a terminal end. In aspects, the smAKAP is linked to two VHH antibodies, wherein a first VHH is at a C-terminus and the second VHH is at an N-terminus. In aspects, the recombinant Spirulina expresses a 5HVZ. In aspects, the 5HVZ is linked to two VHH antibodies in a homodimeric configuration.

Provided are polynucleotide sequences comprising a mutated small-membrane A-kinase anchoring protein (smAKAP) sequence, wherein the mutation comprises one or more residue substitutions as compared to a WT smAKAP sequence.

Provided are vectors comprising disclosed polynucleotide sequences

Provided are also methods of making a recombinant Spirulina comprising contacting a Spirulina cell with vectors of the disclosure.

Provided are pharmaceutical compositions comprising recombinant Spirulina and one or more excipients.

Provided are kits comprising: recombinant Spirulina, polynucleotide sequences, vectors, or pharmaceutical compositions, and instructions for use thereof.

Provided are methods of treatment comprising administering pharmaceutical compositions of the disclosure to a subject in need thereof, wherein the subject comprises a bacterial or viral infection.

Provided are also vectors comprising a nucleotide sequence encoding a polypeptide of SEQ ID NO: 2-7, 14-24, 74, and/or 76. In aspects, a vector comprises from about 80%, 85%, 90%, 95% 96% 97%, 98%, 99%, or 100% identity to SEQ ID NO: 75.

Provided are also recombinant Spirulina comprising vectors of the disclosure.

### BRIEF DESCRIPTION OF THE DRAWINGS

The accompanying figures, which are incorporated herein and form a part of the specification, illustrate some, but not the only or exclusive, example aspects and/or features. It is intended that the aspects and figures disclosed herein are to be considered illustrative rather than limiting.

FIG. 1A-FIG. 1D show protease sensitivity data for constructs: PP917 2xS3b-C8 (FIG. 1A), pp 1895 2xS3b-C8 (FIG. 1B), pp 1895 2xRN-29 (FIG. 1C), and pp 2130 2xRN-29+2xS3b-C8 (FIG. 1D).

FIG. 2 is a graphic depicting hydrophobic residues on smAKAP that are involved in complex formation with 5HVZ.

FIG. 3A is a graphic of the smAKAP peptide that depicts an Arginine residue that is solvent-exposed and can function as a potential Trypsin cleavage site, see SEQ ID NO: 1. Also depicted is the pp 1895 amino acid sequence of SEQ ID NO: 1. FIG. 3B shows normalized frequency of various cuts of the smAKAP peptide. FIG. 3C is a graphic showing additional Lue and His residues on the smAKAP peptide that are potential Trypsin cleavage sites.

FIG. 4 is a graphic of smAKAP depicting exemplary mutations that confer Trypsin and Chymotrypsin resistance.

FIG. 5 shows a western blot of mutant smAKAP clones expressed in an E. coli system.

FIG. 6A-FIG. 6C show Trypsin digestion of clones comprising R63E substitution: pp 2451 (FIG. 6A), pp 1895 (FIG. 6B), and pp 2451 (FIG. 6C). The R63 substitution confers Trypsin resistance in smAKAP peptides.

FIG. 7 shows sizing based complex formation analysis of smAKAP and 5HVZ dimers.

FIG. 8 shows results of a complex formation analysis utilizing mutant constructs pp 2451 and pp 2456 exposed to trypsin and chymotrypsin digestion at 0.1 mg/mL and 0.01 mg/mL.

FIG. 9 shows results of a chymotrypsin resistance assay utilizing clones pp 2451, pp 1895, pp 2455, pp 2456, pp 2454, pp 2452, and pp 2453.

FIG. 10 shows results of a complex formation assay utilizing clones pp 2451, pp 1895, pp 2456, pp 2454, pp 2452, and pp 2453.

FIG. 11 shows an SDS-PAGE gel of a comparative analysis of smAKAP mutant scaffolds.

FIG. 12 shows an SDS-PAGE gel showing that smAKAP linker containing constructs express well in Spirulina.

FIG. 13 shows an SDS-PAGE gel 0 day, 4 days and 7 days after purification of pp 1895. The purified protein show increased degradation and instability following storage at 4 C. This stands in contrast to results from FIG. 11 showing greater stability.

FIG. 14 shows results of a complex formation analysis utilizing mutant construct pp 6510.

### DETAILED DESCRIPTION

Provided are compositions comprising recombinant Spirulina. Provided are also scaffolds comprising binding domains expressed by recombinant Spirulina and methods of improving the same for the manufacture of orally delivered therapeutic proteins. For example, homomeric and heteromeric scaffolds comprising VHH antibodies linked by way of an smAKAP linker can be utilized to increase binding affinity as compared to VHH antibody alone.

Also provided are methods comprising administering the recombinant Spirulina to a subject in need thereof to prevent, treat, or ameliorate an infection, disease, or condition.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a,” “an,” and “the” include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, the term “subject” refers to any subject, e.g., a human or a non-human mammal, for whom diagnosis, prognosis, or therapy is desired. The term “subject” may mean a human or non-human mammal affected, likely to be affected, or suspected to be affected with a disease. The terms “subject” and “patient” are used interchangeably herein. In aspects, a subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile, fish, or the like.

As used herein, the term “a subject in need thereof” includes subjects that could or would benefit from the methods described herein. Subjects in need of treatment include, without limitation, those already with the condition or disorder, those prone to having the condition or disorder, those in which the condition or disorder is suspected, as well as those in which the condition or disorder is to be prevented, ameliorated, or reversed.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

Antibody fragments include the antigen-binding portion of an antibody (i.e., “antigen-binding fragment”), including, inter alia, fab′, F(ab′) 2, fv, domain antibodies (dAb), complementarity Determining Region (CDR) fragments, CDR-grafted antibodies, single chain antibodies (scFv), single chain antibody fragments, chimeric antibodies, diabodies, triabodies, tetrabodies, minibodies, linear antibodies; chelating recombinant antibodies, triple-chain antibodies (tribodies) or diabodies (bibodies), intrabodies (intrabodies), nanobodies (nanobodies), small Modular Immunopharmaceuticals (SMIPs), antigen-binding domain immunoglobulin fusion proteins, single domain antibodies (including camelized antibodies), antibodies containing VHH, or variants or derivatives thereof, and polypeptides containing at least a portion of an immunoglobulin sufficient to bind a specific antigen to a polypeptide (e.g., one, two, three, four, five, or six CDR sequences) so long as the fragment retains the desired biological activity.

### Scaffolds

Provided herein are scaffolds. In aspects, a scaffold can be monomeric, dimeric, trimeric, tetrameric, pentameric, hexameric, heptameric, octameric, nonameric, or decameric. Scaffolds are utilized to multimerize heterologous moieties of the disclosure for expression in Spirulina. For example, a scaffold can be utilized to multimerize a VHH for expression in Spirulina. Multimerization can achieve increased target binding affinity as compared to affinity of a single monomeric VHH or as compared to a scaffold with reduced multimerization (e.g., a trimeric vs. dimeric scaffold).

In aspects, a scaffold is monomeric. A monomeric scaffold can comprise a heterologous moiety attached or linked to a scaffold. In aspects, a scaffold comprises a maltose-binding protein (MBP). In aspects, scaffold comprises thioredoxin (TRX).

In aspects, a scaffold is dimeric. A dimeric scaffold can comprise 5HVZ.

In aspects, a scaffold is trimeric. A trimeric scaffold can comprise cTRP. A trimeric scaffold can comprise SP651.

In aspects, a scaffold is pentameric. A pentameric scaffold can comprise SP737.

In aspects, a scaffold is heptameric. A heptameric scaffold can comprise 4B0F.

In aspects, a scaffold is selected from the group consisting of: oligomerization domain of C4b-binding protein (C4BP), cholera toxin b subunit, oligomerization domains of extracellular matrix proteins, TRX, 5HVZ, cTRP, SP651, SP737, 4B0F, smAKAP, and combinations thereof.

In aspects, any of the scaffolds of the disclosure can be mutated to improve performance. In aspects, a mutation is made to reduce protease sensitivity (e.g., trypsin or chymotrypsin).

smAKAP

In aspects, a scaffold comprises an smAKAP peptide. smAKAP is a small (i.e. 11 kDa) PKA-RI-specific protein kinase A-anchoring protein, referred to herein to as the small-membrane AKAP (smAKAP). smAKAP is tethered to the plasma membrane through a dual acylation of its N-terminal Met-Gly-Cys-motif (myristoylation and palmitoylation, respectively). In aspects, smAKAP is expressed in Spirulina as part of a scaffold of the disclosure. A smAKAP can serve as a linker in a scaffold of the disclosure.

Scaffolds provided can further comprise a 5HVZ domain. In aspects, 5HVZ can spontaneously assemble into dimers inside the Spirulina cell. In aspects, provided is a homodimeric protein containing two protein molecules (5HVZ-VHH and 5HVZ-VHH). In aspects, a scaffold contains maltose binding protein (MBP) on one end and a VHH on the other end. In this configuration the homodimer has two VHHs associated with each other. Dimeric VHHs often allow for tighter binding to their target, in a phenomenon known as avidity. This strategy can result in four associated copies of the VHH, if the VHH is at both ends of the 5HVZ scaffold.

In addition, smAKAP is useful because it spontaneously forms a trimeric complex with the 5HVZ homodimer. This allows for further increases in the multimericity of the scaffolded complex. For instance, depending upon the particular design these trimers can have 3, 4 or 6 VHHs per trimeric complex. Furthermore, the smAKAP can also have a VHH that is different from the one fused to 5HVZ, which allows for combinatorial assembly of different complexes containing unique VHH pairs.

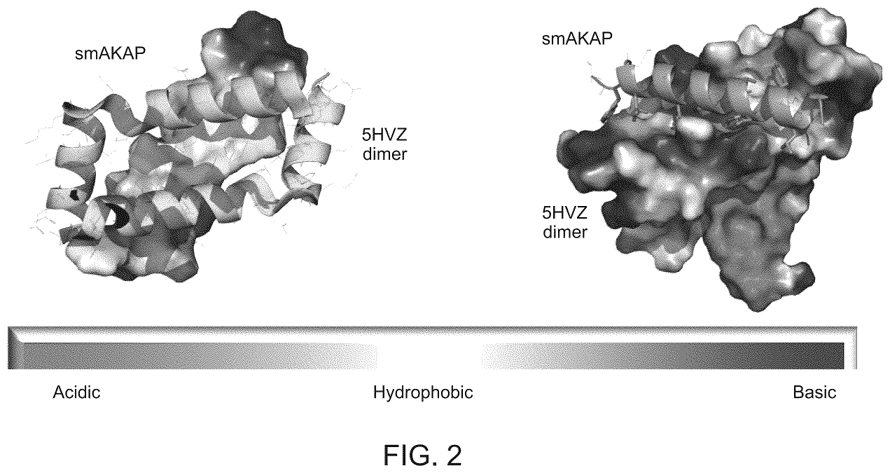

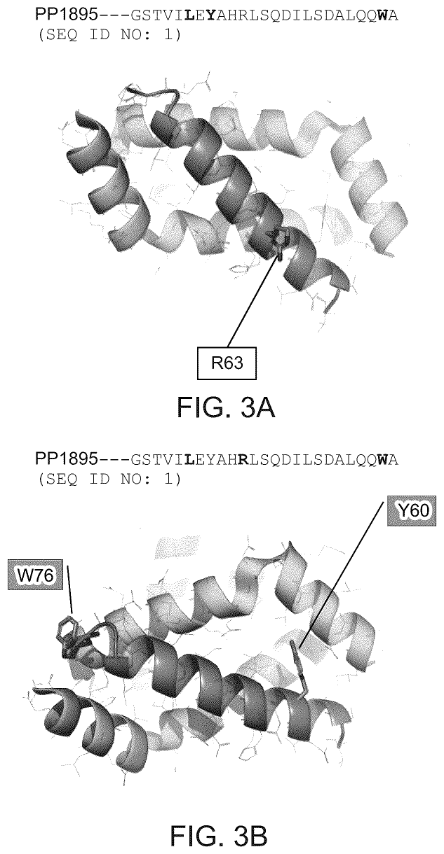

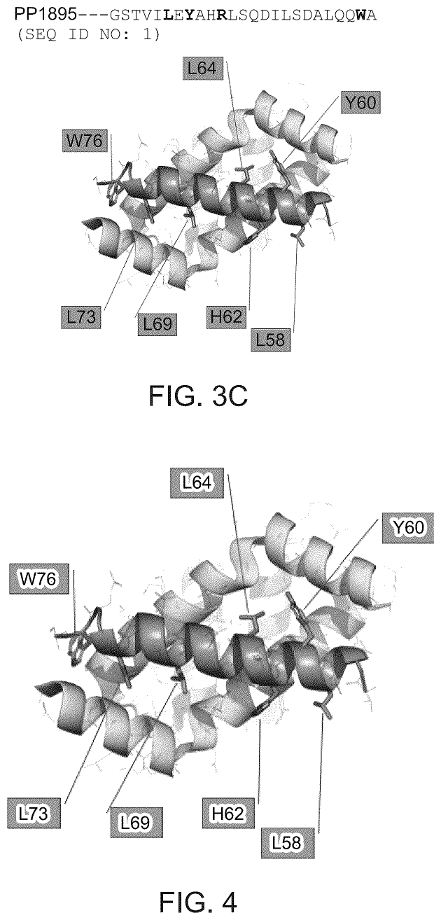

In scaffolds comprising smAKAP and 5HVZ (e.g., Cerberbodies and/or Hydrabodies), the hydrophobic residues on smAKAP are involved in complex formation with 5HVZ, see FIG. 2. In aspects, an smAKAP peptide comprises residues that may be sensitive to protease cleavage see FIG. 3B and Keil, B. Specificity of proteolysis. Springer-Verlag Berlin-Heidelberg-New York, pp. 335. (1992), incorporated by reference in its entirety. Therefore, also provided are compositions comprising mutant smAKAP peptides showing resistance or reduced cleavage by proteases.

To generate mutant smAKAP peptides to confer Trypsin and Chymotrypsin resistance any methodology may be utilized. In aspects, a trypsin sensitive site is removed. In aspects, residues may be replaced such that sites are more basic as compared to WT or more acidic as compared to WT. Exemplary residues that can be mutated are shown in FIG. 4 however, any other residues may also be mutated. In aspects, one or more residues are mutated. In aspects, peptides with two or more mutations show increased resistance to proteases as compared to peptides with single residue mutations. In aspects, combining different hydrophobic mutations improves resistance to proteases.

In aspects, an smAKAP peptide comprises a mutation selected from the group consisting of: C16S, C24S, E5D, Y6H, W22S, C24G, L4E, R9E, LAI, L10I, L19I, and combination thereof of SEQ ID NO: 2. In aspects, the mutant smAKAP comprises mutations at C16S and C24S as compared to SEQ ID NO: 2. In aspects, the mutant smAKAP comprises mutations at C16S E5D, Y6H, C16S, W22S, and C24G as compared to SEQ ID NO: 2. In aspects, the mutant smAKAP comprises mutations at C16S, L4E, E5D, Y6H, R9E, C16S, W22S, and C24G as compared to SEQ ID NO: 2. In aspects, the mutant smAKAP comprises mutations at C16S. L4E, C16S, W22S, and C24G as compared to SEQ ID NO: 2. In aspects, the mutant smAKAP comprises mutations at C16S, R9E, C16S, and C24G as compared to SEQ ID NO: 2. In aspects, the mutant smAKAP comprises mutations at C16S. R9E, C16S, W22S, and C24G as compared to SEQ ID NO: 2. In aspects, the mutant smAKAP comprises mutations at L4I, L10I, C16S, L19I, and C78A as compared to SEQ ID NO: 2. In aspects, the mutant smAKAP comprises mutations at L4I, R9E, L10I, C16S, L19I, W22S, C24G as compared to SEQ ID NO: 2. In aspects, the mutant smAKAP comprises LAI, R9E, C16S, and C24G mutations as compared to SEQ ID NO: 2.

In aspects, a mutant smAKAP comprises an amino acid sequence with at least about 85%, 90%, 95%, 96%, 97%, 98%, 99%, or 100% identity to a sequence of SEQ ID NO: 5, 7, 15, 17, 19, 21, 72, 73, and 74.

In aspects, a mutant smAKAP exhibits increased protease resistance as compared to a WT smAKAP. For example, a composition comprising a mutant smAKAP of the disclosure can exhibit at most about 0.5-fold, 1-fold, 3-fold, 5-fold, 10-fold, 15-fold, 20-fold, 25-fold, 30-fold, 40-fold, 60-fold, 80-fold, 100-fold, 120-fold, 140-fold, 160-fold, 180-fold, 200-fold, 250-fold, 300-fold, or up to 350-fold reduced activity and/or cleavage events as compared to WT. For example, a composition comprising a mutant smAKAP of the disclosure can exhibit at most about 0.5%, 2%, 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80% reduced activity and/or cleavage events as compared to WT. Activity can be determined following incubation with a protease. For example a mutant smAKAP of the disclosure can exhibited at most about 0.5%, 2%, 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, or 60% reduced activity as compared to a WT smAKAP following a 1 hour incubation with a protease.

In aspects, a scaffold comprises a Cerberbody or Hydrabody complex as described herein and below that comprises a mutant smAKAP peptide.

### Cerberbody

In aspects, a Cerberbody construct can be formed when a Spirulina strain (either two independent strains or a single strain from different ORFs) express a 5HVZ mediated homodimer molecule and smAKAP linked monomer VHH. These independent complexes form a further complex structure that will have three antigen binding sites. In aspects, another heterologous moiety of the disclosure can replace one or more VHHs in a cerberbody. As described herein, any of the scaffolds can be mutated to improve performance. For example, a smAKAP may be mutated as described herein to reduce protease sensitivity.

The presence of increased antigen binding reagents (VHHs) results in increased binding affinity, due to avidity-based interactions of disclosed Cerberbody constructs. The three binding VHHs can be derived from the same VHH forming a homo-trimer. The design also enables mixing diverse VHHs with desirable binding and target neutralization properties. Such a molecule complex will be able to bind multiple epitopes and mutant variants of targets described herein (e.g., pathogens and variants thereof) that may not be captured by conventional immune surveillance or conventional therapies. In aspects, Cerberbody complexes show increased binding when compared to dimeric or monomeric forms. For example, binding may be increased by at least about or at most about: 5-fold, 15-fold, 25-fold, 35-fold, 45-fold, 55-fold, 65-fold, 75-fold, 85-fold, 95-fold, 105-fold, 115-fold, 125-fold, 135-fold, 145-fold, 155-fold, 165-fold, 175-fold, 185-fold, 195-fold, 205-fold, 215-fold, 225-fold, 235-fold, 245-fold, or 300-fold as compared to an otherwise comparable dimeric or monomeric scaffold.

### Hydrabody

In aspects, provided are also Hydrabody complexes. In aspects, a Hydrabody comprises at least four heterologous moieties. In aspects, a Hydrabody comprises at least four antigen binding VHHs.

Hydrabody complexes can be formed when a Spirulina strain (either two independent strains or a single strain from different ORFs) express a 5HVZ mediated homodimer molecule and smAKAP linked dimeric VHHs. The smAKAP mediated dimers contain VHHs on both the N- and C-terminal of smAKAP. The VHHs fused to the smAKAP can form homo-dimeric (the same VHH) or hetero-dimeric (two different VHHs on either end). Such a configuration can enable inclusion of multiple, therapeutically effective VHHs in a single complex.

In aspects, hydrabody complexes show increased binding when compared to trimeric, dimeric, or monomeric forms. For example, binding may be increased by at least about or at most about: 5-fold, 15-fold, 25-fold, 35-fold, 45-fold, 55-fold, 65-fold, 75-fold, 85-fold, 95-fold, 105-fold, 115-fold, 125-fold, 135-fold, 145-fold, 155-fold, 165-fold, 175-fold, 185-fold, 195-fold, 205-fold, 215-fold, 225-fold, 235-fold, 245-fold, or 300-fold as compared to an otherwise comparable trimeric, dimeric, or monomeric scaffold.

In addition to increased avidity-based apparent binding, the higher order complexes of Cerberbody and Hydrabody enable the formation of super potent and cross-reactive therapeutic complexes that can bind and neutralize diverse targets. For example, in aspects, a scaffold of the disclosure is administered to a subject in need thereof to neutralize two or more targets. For example, a Hydrabody that contains a dimer of dimers of two VHHs that bind and neutralize single targets can bind better than each separately and neutralize both targets. Such a design combines multiple components easily where a diverse epitope can be engaged resulting in decreased target escape (e.g., viral escape).

### Heterodimer

In aspects, a version of the 5HVZ scaffold forms obligate heterodimers instead of homodimers. This allows for the assembly of dimeric 5HVZ complexes containing four different functional domains. In aspects, the four different functional domains comprise two different functional domains associated with each of the 5HVZ scaffolds.

In aspects, provided herein are heterodimer complexes. In aspects, the heterodimer complex comprises a dimeric 5HVZ complex. In aspects, the dimeric 5HVZ complexes comprise at least two, three, four, five or six different functional domains. In aspects, the dimeric 5HVZ complexes comprise at least four different functional domains. In aspects, the dimeric 5HVZ complexes comprise four different functional domains. In aspects, the dimeric 5HVZ complexes comprise the same functional domains.

In aspects, the functional domain comprises functional domains associated with each of the 5HVZ scaffolds. In aspects, the functional domain comprises at least one, two, three, four, five or six functional domains associated with each of the 5HVZ scaffolds. In aspects, the functional domain comprises at least two functional domains associated with each of the 5HVZ scaffolds. In aspects, the functional domain comprises two functional domains associated with each of the 5HVZ scaffolds. In aspects, the functional domains associated with each of the 5HVZ scaffolds are the same. In aspects, the functional domains associated with each of the 5HVZ scaffolds are different. In aspects, the dimeric 5HVZ complexes comprise at least two, three, four, five or six different functional domains associated with each of the 5HVZ scaffolds. In aspects, the dimeric 5HVZ complexes comprise at least two different functional domains associated with each of the 5HVZ scaffolds. In aspects, the dimeric 5HVZ complexes comprise two different functional domains associated with each of the 5HVZ scaffolds. In aspects, the dimeric 5HVZ complexes comprise four different functional domains, wherein two different functional domains are associated with each of the 5HVZ scaffolds. In aspects, the dimeric 5HVZ complexes comprise six different functional domains, wherein three different functional domains are associated with each of the 5HVZ scaffolds. In aspects, the dimeric 5HVZ complexes comprise eight different functional domains, wherein four different functional domains are associated with each of the 5HVZ scaffolds.

In aspects, the 5HVZ scaffolds comprise a functional domain comprising 1-5HVZ1-domain 2. In aspects, the 5HVZ scaffold comprises a functional domain comprising 3-5HVZ-domain 4. In aspects, the 5HVZ scaffold comprises a functional domain comprising 1-5HVZ1-domain 2 and a functional domain comprising 3-5HVZ-domain 4.

In aspects, heterodimer complexes show increased binding in comparison to monomeric forms. In aspects, binding may be increased by at least about or at most about: 5-fold, 15-fold, 25-fold, 35-fold, 45-fold, 55-fold, 65-fold, 75-fold, 85-fold, 95-fold, 105-fold, 115-fold, 125-fold, 135-fold, 145-fold, 155-fold, 165-fold, 175-fold, 185-fold, 195-fold, 205-fold, 215-fold, 225-fold, 235-fold, 245-fold, or 300-fold as compared to an otherwise comparable monomeric scaffolds.

In addition to increased avidity-based apparent binding, the heterodimers enable the formation of super potent and cross-reactive therapeutic complexes that can bind and neutralize diverse targets. For example, in aspects, a scaffold of the disclosure is administered to a subject in need thereof to neutralize two or more targets.

### Heterologous Moiety

In aspects, a scaffold is bound to a heterologous moiety. In aspects, a heterologous moiety comprises a therapeutic. In aspects, a therapeutic comprises a biologic. In aspects, a therapeutic comprises a binding agent. In aspects, a heterologous moiety is linked to a scaffold protein at the N-terminus or the C-terminus, or in the body of the scaffold protein.

In aspects, a therapeutic is a binding agent that comprises an antibody or functional fragment thereof. In aspects, a binding agent comprises an isolated fully human, humanized or chimeric antibody or a fragment thereof. In aspects, a binding molecule is an antibody fragment or a single variable domain antibody, preferably a Fab, a Fab′, a F(ab′) 2, a scFv, a dAb or a VHH.

In aspects, a heterologous moiety comprises a binding agent that is a VHH. VHHs can be produced economically in unlimited amounts, are more stable when exposed to heat and solvents as compared to conventional antibodies, and are amenable to genetic manipulations for a variety of uses, including scaffolding, labeling, and altering specific amino acids. Further, VHHs are 1/10th the size of conventional antibodies. With their smaller size, VHHs offer higher density of binding domains that provide an outstanding advantage in terms of increased signal and therefore higher sensitivities as compared to conventional antibodies.

An exemplary VHH comprises an antigen binding fragment of a heavy chain only antibody. In aspects, a VHH is from a camelid single-chain antibody. In aspects a VHH can be expressed intracellularly. In aspects, a VHH can be expressed extracellularly. In aspects, a VHH is expressed as a monomer, dimer, trimer, and/or heptamer. In aspects, a VHH is expressed as a monomer. In aspects, a VHH is expressed as a dimer. In aspects, a VHH is expressed as a trimer. In aspects, a VHH is expressed as a heptamer. Expression can be constitutive.

In aspects a VHH targets an antigen associated with a disease or condition. Exemplary diseases or conditions can be autoimmune, metabolic, neurological, cancer, and parasitic, bacterial, viral, and any combination thereof. In aspects, a VHH targets an autoimmune target. An autoimmune target can be associated with a disease selected from the group consisting of: inflammatory bowel disease (IBD), celiac disease, ulcerative colitis, and Crohn's disease. In aspects, a disease or condition is metabolic. Exemplary metabolic disease or conditions can be a cardiometabolic disease (CMD). CMD is a cluster of related conditions that includes obesity, diabetes, and cardiovascular disease. In aspects, a disease or condition is bacterial. A bacterial condition can comprise diarrhea caused by a bacterial pathogen. Exemplary bacterial pathogens comprise E. Coli, Campylobacter jejuni, and enterotoxigenic E. coli.

In aspects, a heterologous moiety is selected from the group consisting of a small molecule (e.g., a drug), a peptide (e.g., ligand), and a nucleic acid (e.g., siRNA, DNA, modified RNA, RNA). In aspects, a heterologous moiety possesses at least one effector activity selected from the group consisting of: modulates a biological activity, binds a regulatory protein, modulates enzymatic activity, modulates substrate binding, modulates receptor activation, modulates protein stability/degradation, and modulates transcript stability/degradation. In aspects, a heterologous moiety possesses at least one targeted function selected from the group consisting of: modulates a function, modulates a molecule (e.g., enzyme, protein or nucleic acid), and is localized to a specific location. In aspects, a heterologous moiety is a tag or label, e.g., cleavable. In aspects, a heterologous moiety is selected from the group consisting of: an epigenetic modifying agent, epigenetic enzyme, a bicyclic peptide, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis inhibitor, a nuclease, a protein fragment or domain, a tag, an antigen, an antibody or antibody fragment, a ligand or a receptor, a synthetic or analog peptide from a naturally-bioactive peptide, an anti-microbial peptide, a pore-forming peptide, a targeting or cytotoxic peptide, a degradation or self-destruction peptide, a CRISPR system or component thereof, DNA, RNA, artificial nucleic acids, a nanoparticle, an oligonucleotide aptamer, a peptide aptamer, and an pharmacokinetics or pharmacodynamics (PK/PD) agent.

In aspects, the heterologous moiety is selected from the group consisting of: a drug, a toxin, a cytotoxic agent, an imaging agent, a radionuclide, a radioactive compound, an organic polymer, an inorganic polymer, a polyethylene glycol (PEG), biotin, an albumin, a ligand, a receptor, a binding peptide, an epitope tag, a recombinant polypeptide polymer, a cytokine, and a combination of two or more of said moieties. In aspects, the heterologous moiety is the albumin and the albumin comprises human serum albumin. In aspects, the heterologous moiety is the antibody or the fragment thereof and comprises a domain of an antibody, an antibody fragment, a single chain antibody, a domain antibody, or any combination thereof.

In aspects, the heterologous moiety comprises an antibody. In aspects, the antibody is selected from the group consisting of: an Fc domain of an antibody, an antibody fragment, and a single chain antibody.

In aspects, a scaffold of the disclosure can comprise a heterologous moiety of the disclosure. In aspects, a heterologous moiety comprises a VHH. In aspects, a Cerberbody or Hydrabody can comprise multiple VHHs of the same sequence or different sequences.

VHHs derived from camelid single-chain antibodies are ideal for expression in prokaryotes, like Spirulina, because neither intracellular formation of disulfide bonds nor specific glycosylation is needed for synthesis of the bioactive protein. In aspects, a VHH is constitutively expressed in Spirulina. Expression can be modulated utilizing any promoter. In aspects, a promoter comprises Pepc600. As described herein, a VHH can be expressed on a scaffold under various formats, including monomers, dimers, trimers and heptamers.

Because they are easily expressed in prokaryotes, VHHs can be rapidly isolated from high diversity, naive phage-display libraries. These typically have mid-nM affinities for their antigen targets, and therefore further mutagenesis can be performed to achieve the higher affinities. Expression of VHHs as high-avidity multimers, as described herein, bypasses this and therefore can accelerate product development.

In aspects, a VHH of the disclosure binds to a virus or portion thereof, including but not limited to, bacteriophage, RNA bacteriophage (e.g. MS2, AP205, PP7 and QB), Helicobacter pylori, infectious hematopoietic necrosis virus (IHNV), parvovirus, Herpes Simplex Virus, Hepatitis A virus, Hepatitis B virus, Hepatitis C virus, Measles virus, Mumps virus, Rubella virus, Human Immunodeficiency Virus (HIV), Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, White Spot Syndrome Virus, a coronavirus, MERS, SARS, and SARS-COV-2.

In aspects, a VHH is an anti-campylobacter VHH. In aspects, the campylobacter is a C. jejuni. In aspects, the VHH binds to a campylobacter component. In aspects, the VHH binds flagellin. In aspects, administration increases Campylobacter shedding. In aspects, administration reduces the levels of biomarkers. In aspects, the biomarker is an inflammation biomarker.

In aspects, the recombinant Spirulina comprises a VHH that binds to an anti-Clostridium toxin. In aspects, the Clostridium is C. difficile. In aspects, the VHH binds to a Clostridium component, toxin A, or toxin B. In aspects, the VHH comprises the amino acid sequence of any of SEQ ID NOs: 25-37 or fragment thereof.

In aspects, the recombinant Spirulina comprises an anti-Campylobacter VHH. In aspects, the campylobacter is a C. jejuni. In aspects, the VHH binds to a campylobacter component. In aspects, the VHH binds flagellin. In aspects, administration increases Campylobacter shedding. In aspects, administration reduces the levels of biomarkers. In aspects, the biomarker is an inflammation biomarker.

In aspects, the recombinant Spirulina comprises a polypeptide or fragment thereof that binds to a Norovirus polypeptide or antigen. In aspects, the recombinant Spirulina comprises a polypeptide or fragment thereof that binds to a norovirus P domain. In aspects, the polypeptide or fragment thereof is a VHH. In aspects, the recombinant Spirulina comprises a VHH that binds to a Norovirus polypeptide. In aspects, the recombinant Spirulina comprises a VHH that binds to a norovirus P domain. In aspects, the recombinant Spirulina comprises a polypeptide or fragment thereof that binds to a GII genotype, a G1 genotype, a G11.10 genotype. In aspects, the recombinant Spirulina comprises a polypeptide or fragment thereof that binds to a polypeptide from two or more norovirus genotypes. In aspects, the recombinant Spirulina comprises a VHH comprising a Nano85 nanobody, a Nano26 nanobody, a Nano94 nanobody, a K922 antibody or a modified sequence or fragment thereof. In aspects, the recombinant Spirulina comprises a VHH comprising Nano85 and/or a loop grafted modification thereof.

In aspects, a Spirulina comprises a scaffold comprising a VHH that binds to a malaria polypeptide or antigen or fragment thereof. In aspects, the malaria comprises the genus Plasmodium. In aspects, the malaria comprises Plasmodium falciparum, P. vivax, P. ovale curtisi, P. ovale wallikeri, P. malariae or P. knowlesi. In aspects, the malaria is P. falciparum. In aspects, the recombinant Spirulina comprises a polypeptide that binds to a malaria associated protein of fragment thereof in the liver or in the blood. In aspects, the recombinant Spirulina comprises a polypeptide that binds to a malaria associated protein or fragment thereof during the exoerythrocytic cycle, rupture of the hepatocytes, or the erythrocytic cycle. In aspects, the recombinant Spirulina comprises a polypeptide that binds to a malaria associated protein or fragment thereof during the exoerythrocytic cycle. In aspects, the recombinant Spirulina comprises a polypeptide that binds to a malaria associated protein or fragment thereof derived from a stage comprising sporozoites, merozoites, trophozoites, schizont or gametocytes. In aspects, the recombinant Spirulina comprises a polypeptide that binds to a malaria associated protein or fragment thereof on the surface of sporozoites. In aspects, the recombinant Spirulina comprises a polypeptide that binds to a Circumsporozoite protein (CSP) or fragment thereof. In aspects, the recombinant Spirulina comprises a polypeptide that binds to a P. falciparum Circumsporozoite protein (PfCSP) or fragment thereof. In aspects, the binding disclosed herein can block hepatocyte infection by sporozoites and protect against malaria. In aspects, the VHH binds one or more NANP repeats. In aspects, a VHH binds a CSP polypeptide. In aspects a VHH binds a polypeptide comprising one or more NANP repeats.

Exemplary VHHs of the disclosure are provided in Table 0. In aspects, a scaffold of the disclosure is linked to a VHH comprising at least about 85%, 90%, 95%, 96%, 97%, 98%, 99%, or 100% identity to a VHH of Table 0. In aspects, a scaffold of the disclosure is linked to a VHH comprising at least about 85%, 90%, 95%, 96%, 97%, 98%, 99%, or 100% identity to SEQ ID NO: 25-67. In aspects, a scaffold of the disclosure is linked to a VHH comprising at least about 85%, 90%, 95%, 96%, 97%, 98%, 99%, or 100% identity to SEQ ID NO: 25-37. In aspects, a scaffold of the disclosure is linked to a VHH comprising at least about 85%, 90%, 95%, 96%, 97%, 98%, 99%, or 100% identity to SEQ ID NO: 38-67.

TABLE 0
Exemplary VHH sequences

| | SEQ ID | |
|---|---|---|
| VHH | No | Sequence |
| C. difficile | | |
| Anti-tcdB | 25 | QVQLVESGGGLVQPGGSLRLSCEASGFTLDYYGIGWFRQPPG |
| VHH 5D | | KEREAVSYISASARTILYADSVKGRFTISRDNAKNAVYLQMN |
| | | SLKREDTAVYYCARRRFSASSVNRWLADDYDVWGRGTQVA |
| | | VSSEPKTPKPQ |
| Anti-tcdB | 26 | QVQLVESGGGLVQTGGSLRLSCASSGSIAGFETVTWSRQAPG |
| VHH E3 | | KSLQWVASMTKTNNEIYSDSVKGRFIISRDNAKNTVYLQMNS |
| | | LKPEDTGVYFCKGPELRGQGIQVTVSSEPKTPKPQ |
| Anti-tcdB | 27 | VQLVESGGGLVQAGGSLRLSCAASGLMFGAMTMGWYRQAP |
| VHH 5E | | GKEREMVAYITAGGTESYSESVKGRFTISRINANNMVYLQMT |
| | | NLKVEDTAVYYCNAHNFWRTSRNWGQGTQVTVSSEPKTPKP |
| Anti-tcdB | 28 | VQLVESGGGLVQAGDSLTLSCAASESTFNTFSMAWFRQAPGK |
| VHH B12 | | EREYVAAFSRSGGTTNYADSVKGRATISTDNAKNTVYLHMN |
| | | SLKPEDTAVYFCAADRPAGRAYFQSRSYNYWGQGTQVTVSS |
| | | AHHSEDP |
| Anti-tcdB | 29 | VQLVESGGGSVQIGGSLRLSCVASGFTFSKNIMSWARQAPGK |
| VHH A11 | | GLEWVSTISIGGAATSYADSVKGRFTISRDNANDTLYLQMNN |
| | | LKPEDTAVYYCSRGPRTYINTASRGQGTQVTVSSEPKTPKP |
| Anti-tcdB | 30 | VQLVESGGGLVQAGGSLRLSCAAPGLTFTSYRMGWFRQAPG |
| VHH A1 | | KEREYVAAITGAGATNYADSAKGRFTISKNNTASTVHLQMNS |
| | | LKPEDTAVYYCAASNRAGGYWRASQYDYWGQGTQVTVSSA |
| | | HHSEDP |
| Anti-tcdB | 31 | QVQLVESGGGLVQPGGSLRLSCAASGFSLDYYGIGWFRQAPG |
| VHH 2D | | KERQEVSYISASAKTKLYSDSVKGRFTISRDNAKNAVYLEMN |
| | | SLKREDTAVYYCARRRFDASASNRWLAADYDYWGQGTQVT |
| | | VSSEPKTPKPQ |
| Anti-tcdB | 32 | QVQLVESGGGLVQAGGSLRLSCVSSERNPGINAMGWYRQAP |
| VHH 2Ds | | GSQRELVAIWQTGGSLNYADSVKGRFTISRDNLKNTVYLQM |
| | | NSLKPEDTAVYYCYLKKWRDQYWGQGTQVTVSSEPKTPKPQ |
| Anti-tcdB | 33 | QVQLVESGGGLVEAGGSLRLSCVVTGSSFSTSTMAWYRQPPG |
| VHH 7F | | KQREWVASFTSGGAIKYTDSVKGRFTMSRDNAKKMTYLQM |
| | | ENLKPEDTAVYYCALHNAVSGSSWGRGTQVTVSSEPKTPKPQ |
| Anti-tcdB | 34 | VQLVESGGGLVQAGDSLTLSCAASESTFNTFSMAWFRQAPGK |
| VHH B12 | | EREYVAAFSRSGGTTNYADSVKGRATISTDNAKNTVYLHMN |
| | | SLKPEDTAVYFCAADRPAGRAYFQSRSYNYWGQGTQVTVSS |
| | | AHHSEDP |
| Anti-tcdB | 35 | VQLVESGGGLVQAGGSLRLSCVGSGRNPGINAMGWYRQAPG |
| VHH AB8 | | SQRELVAVWQTGGSTNYADSVKGRFTISRDNLKNTVYLQMN |
| | | SLKPEDTAVYYCYLKKWRDEYWGQGTQVTVSSAHHSEDP |
| Anti-tcdB | 36 | VQLVESGGGLVQAGESLRLSCVVSESIFRINTMGWYRQTPGK |
| VHH C6 | | QREVVARITLRNSTTYADSVKGRFTISRDDAKNTLYLKMDSL |
| | | KPEDTAVYYCHRYPLIFRNSPYWGQGTQVTVSSEPKTPKP |
| Anti-tcdB | 37 | VQLVESGGGLVQAGESLRLSCVVSESIFRINTMGWYRQTPGK |
| VHH C12 | | QREVVARITLRNSTTYADSVKGRFTISRDDAKNTLYLKMDSL |
| | | KPEDTAVYYCHRYPLIFRNSPYWGQGTQVTVSSEPKTP |
| Norovirus | | |
| N40 | 38 | QVQLQESGGGLVQPGGSLRLSCAASESTPSINTMGWYRQAPG |
| | | KERELVATITSGGMTNYADSVKGRFTISRDNGKNTVYLQMNS |
| | | LEPGDTAVYYCNLKRRDLQARFGGYWGQGTQVTVSSEPKTQ |
| | | TTTSGR |
| N41 | 39 | QVKLQQSGGGLVQPGGSLRLSCAASESTISINTLGWYRQAPG |
| | | NQRELVATITTGGTTNYADSVKGRFTISRDNAKNTVYLQMNN |
| | | LEPGDTAVYYCNLKRRDLQSRFGGYWGQGTQVTVSSEPQDT |
| | | KTTTSGR |
| N42 | 40 | QVQLQQSGGGLVQPGGSLRLSCVASESTVSINIMGWYRQAPG |
| | | KQRELVATITTGGTTNYADSVKGRFTISRDNAKNTVYLQMNS |
| | | LEPEDTAVYYCNLKRRDLQARFGGYWGQGTQVTVSSEPKTP |
| | | KPTSGR |
| N43 | 41 | QVKLQQSGGGLVQPGGSLRLSCAASESTPSINTMGWYRQAPG |
| | | KERELVATITSGGMTNYA |
| | | DSVKGRFTISRDNGKNTVYLQMNSLEPGDTAVYYCNLKRRD |
| | | LQARFGGYWGQGTQVTVSS |
| | | EPKTPKPQSGR |
| N44 | 42 | QVKLQQSGGGLVQPGGSLRLSCAASGFTFRNYAMSWVRQAP |
| | | GKGLEWVSAIAAGGAVTKYADSVKGRFTISRDNARDTLYLQ |
| | | MNSLKPEDTAVYYCAKPRDFWYSPEFDFRGQGTQVTVSSEPK |
| | | NQNQTSGR |
| N45 | 43 | QVQLQQSGGGLVQAGDSLTISCASSLFTFSTSTMGWFRQAPG |
| | | KEREFVAAIKSSGSSMYYADSVQGRFTISRDNAKKTVTLQMN |
| | | SLKPEDTAVYYCAKGVYGSRRSADFGSWGQGTQVTVSSEPK |
| | | TPKPQSGR |
| N46 | 44 | QVQLQQSGGGLVQAGDSLRISCAASLFTFSTSTMGWFRQAPG |
| | | KEREFVAAIRSTGDSMYYADSVQGRFTISRDNAKKMVYLQM |
| | | NNLKPEDTAVYYCAKGVYGSRRSADFGSWGQGTQVTVSSEP |
| | | KTPKPQSGR |
| N47 | 45 | DVQLQASGGGLVQAGGSLRISCTADGYTFSTSTMGWFRQAP |
| | | GKEREFVAAIKSDGSIMYYADSVAGRFIISRDNAKKMVFLQM |
| | | DRLKPEDTAVYYCAKGVYGSRRSADFGWWGQGTQVTVSSE |
| | | PKTPKPQSGR |
| N48 | 46 | QVKLQESGGGLVQAGESLRLSCAASGSNFSINGVGWYRQAP |
| | | GKQRELVAGITNGGYTSYADSVKGRFTISTDNAKNTVYLQM |
| | | NSLRPEDTAVYYCNANFQIHRSGADYVRNYWGQGTQVTVSA |
| | | EPRTKTTTSGR |
| N49 | 47 | QVKLQQSGGGLVQAGGSLRLSCAASGNFFTLNGVAWYRQAP |
| | | GKQRELVAGITSGGWTNYADSVKGRFTISADNAKNTVYLQM |
| | | NSLRPEDTAVYYCNANLQIHRDSSGDVRNVWGQGTQVTVSS |
| | | EPKTPKPQSGR |
| N50 | 48 | DVQLQASGGGLVQAGGSLRLSCAASGSFFSINGVGWYRQAP |
| | | GKQRELVAGITNGGFTNYADSVKGRFTISRDNAKNTVYLQM |
| | | NSLKPEDTAVYYCNANLQISRSEDGAYVVRNYWGQGTQITV |
| | | SSEPKTPKPQSGR |
| N51 | 49 | DVQLQASGGGLVQAGGSLRLSCAASGSGFSINGVGWYRQTP |
| | | GRQRELVAGITIGGYTNYADSVKGRFTISSDNAKNTVYLQMN |
| | | SLKPEDTAVYYCNANLQFYRGGGSDVKNYWGQGTQVTVSSE |
| | | PKTPKPQSGR |
| N52 | 50 | QVQLQESGGGLVQAGGSLRLSCAASGLTFSSYAMGWFHQAP |
| | | GKEREFVAAINWSGRDTYYADSVKGRFTISRDNRKNTVYLQ |
| | | MNSLKPEDTAVYYCAAAEFFSSGDPLPGMDYWGKGTLVTVS |
| | | SEPKTQNHNSGR |
| N53 | 51 | QVKLQQSGGGLVQAGGSLRLSCAASGLTFSSYAMGWFRQAP |
| | | GKEREFVAAINWSGRDTYYADSVKGRFTISRDNAKNTVYLQT |
| | | NSLKPEDTAVYYCAAAEFLPTQRSPREYDYWGLGTQVTVSSE |
| | | PKTPKPQSGR |
| N54 | 52 | QVKLQQSGGGLVQAGGSLRLSCAASGYAFNTYTMAWFRQA |
| | | PGKEREFVALVGMKVDGKIYADSVKGRFTISRDNEQKTVLLE |
| | | MNHLEPEDTAIYYCAASRRFWTAALNGADYPYWGQGTQVT |
| | | VSSEPKTPKPQSGR |
| N55 | 53 | QVKLQQSGGGLVQAGGSLRLSCAASGIVESFNAMGWYRVPP |
| | | GKQRELVADILKSGGTNVVDSVKGRFAISRDSAQNTLYLQMN |
| | | RLKPEDTAVYYCNARDWSDGFDEYWGQGTQVTVSSEPKTPK |
| | | PQSGR |
| N56 | 54 | QVQLQQSGGGLVQAGGSLRLSCSASGRTFSNYVMGWFRQAP |
| | | GKEREFVATISASGGSTYCADSVEGRFTISRDNAKNTAYLQM |
| | | NNLEPEDTAVYYCASGPRANASIRRSGYNYWGQGTQVTVSS |
| | | EPKTPKPQSGR |
| N57 | 55 | QVQLQQSGGGLVQAGGSLRLSCAASGLAFSSYAITWLRQAPG |
| | | TEREFVALISGSGSSTYYADSVKGRFTISRNNAKNTVYLQMNS |
| | | LKPEDTAVYYCTASEFLLHPPPPNQKYDYWGQGTQVTVSSEP |
| | | KTPKHTSGR |
| N58 | 56 | QVQLQQSGGGLVRAGGSLRLSCSASGRTFGNEVMGWFRQAP |
| | | GKEREFVAAINWSSGNTYYRDSVKGRFTISRDNAKNTVYLQ |
| | | MNSLEPEDTAVYYCAARSRPAISTRRPDYFAWGQGTQVTVSS |
| | | EPKTPKPQSGR |
| N59 | 57 | QVQLQQSGGGLVQAGGSLRLSCTASGRIDRTYTVSWFRQGPG |
| | | KEREFVATISWDGSIYYDNAVEGRFSISGDNAKTTVALQMNS |
| | | LKPEDTAVYYCAARRRVFSRAAAAYNYWGQGTQVTVSSEPK |
| | | TPKPQSGR |
| N60 | 58 | QVKLQQSGGGLVQPGGSLRLTCAASGFPFSTYAIRWVRRPPG |
| | | KGLEWVSTIHPDFTTNYADSVSRRFTISRDNAKNTVYLQMNS |
| | | LKPEDTAVYYCSRGVSGERGQGTQVTVSSEPKTPKPHSGR |
| N61 | 59 | QVQLQQSGGGLVQPGGSLRLSCAASGSIFSIHTMGWYRQAPG |
| | | KQRELVTTITTGGTTNYADSVKGRFTISRDDAKNTVYLQMNN |
| | | LKPEDTAVYYCYALIQTASTTWYRQYWGQGTQVTVSSEPKT |
| | | QNHNSGR |
| N62 | 60 | QVKLQESGGGLVQPGGSLRLSCTASRSIFTRAMAWYRQAPGK |
| | | QRELVAAIDSGDRTHYADSVKGRFTISRNNAKDTLYLQMNSL |
| | | KSEDTAVYYCNANLGALLDYWGQGTQVTVSSEPKTQTTTSG |
| | | R |
| N63 | 61 | QVKLQESGGGLVQAGGSLRLSCAASGFTFATYAMAWYRQAP |
| | | GKQRELVASISNFGSTAYGDSVKGRFTISRDNAKNTVYLEMN |
| | | SLKSEDTAVYYCKRVRDVIGRPELWGQGTQVTVSSEPKTPKP |
| | | HSGR |
| N64 | 62 | QVKLQESGGGLVQPGGSLRLSCLPSINIFSLAAMGWYRQAPG |
| | | KQRELVASISSGGTANYADSYADSVKGRFTISRDIAKNTVDLQ |
| | | MNSLKPEDTAVYYCKVDSYTYGTDIWGKGVLVTVSSEPQDT |
| | | KTTSGR |
| N65 | 63 | QVKLQQSGGGLVQAGGSLRLSCAASGTFSRYAMGWFRQAPG |
| | | KEREFVAAINWTGGSTYYADSVKGRFTISGDNAKNTVYLQM |
| | | NSLKPGDTAVYYCAAEVHPGDYGLTYMQSQYEYDYWGQGT |
| | | QVTVSSEPKTPKPTTSGR |
| N66 | 64 | DVQLQASGGGLVQAGGSLRLSCAASTSRFSSYAMGWSRQAP |
| | | GKQRELVASISSSGLTTNYADSVKGRFTISRDNAKNTVYLQM |
| | | NSLKPEDTAVYYCKADGRRYSLNEYWGQGTQVTVSSEPKTP |
| | | KPQPSGR |
| N67 | 65 | DVQLQASGGGLVQAGGSLRLSCAASGSTISSYAMAWYRQAP |
| | | GKRRELVAHISSGGSTNYADSVKGRFTISRDNAKNTVYLQMN |
| | | SLKPEDTAVYYCNIYYGGDYYYTGVKPNPWGQGTQVTVSSA |
| | | HHSEDPRGR |
| N68 | 66 | QVKLQESGGGWVQAGGSLRLSCAASALTASITTMGWFRQTP |
| | | EKEREFLAAINWTGDYKYYADSVKGRFTISRDNAKNTVDLQ |
| | | MNQLKPEDTAVYYCAASKIRNDIYLNDYTWYQYWGQGTQV |
| | | TVSSEPKTPKPQSGR |
| N69 | 67 | DVQLQASGGGLVQAGGSLRLSCVASARTFSSYAMGWFRQAP |
| | | GKEREFVAAISWSGASTDYADSVKGRFTISRDNAKKTVYLQM |
| | | NTLKPEDTAVYYCAAHHITPTGSYYYSEPLPVDMVYDYWGQ |
| | | GTQVTVSSEPKTKTTTSGR |

A scaffold comprising a VHH of the disclosure neutralizes or blocks the activity of a target. In aspects, administration of the present disclosure neutralizes or blocks the activity of the target by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 98%, about 99%, or about 100%.

In aspects, a VHH can be expressed as a fusion protein with a solubility-enhancing chaperone, such as the E. coli maltose-binding protein (MBP).

In aspects, a composition provided herein comprising a scaffold can bind from about 1, 2, 3, 4, 5, 6, 7, or up to about 8 different targets by way of a heterologous moiety. In aspects, a scaffold of the disclosure binds 2 targets. In aspects, a scaffold of the disclosure binds 3 targets. In aspects, a scaffold of the disclosure binds 4 targets. In aspects, a scaffold of the disclosure binds 5 targets. In aspects, a scaffold of the disclosure binds 6 targets. In aspects, a scaffold of the disclosure binds 7 targets. In aspects, a scaffold of the disclosure binds 8 targets.

Also provided are nucleic acids that encode any of the heterologous moieties of the disclosure and/or a polypeptide of the disclosure such as a Cerberbody and/or a Hydrabody. Such a nucleic acid will also be referred to below as a “nucleic acid of the disclosure” and may for example be in the form of a genetic construct.

In aspects, a scaffold of the disclosure, such as a Cerberbody or a Hydrabody, is more effective at neutralizing a target as compared to a monomeric, dimeric, or trimeric construct. In aspects, the effectiveness is at least about 1-fold, 10-fold, 20-fold, 30-fold, 40-fold, 50-fold, 60-fold, 70-fold, 80-fold, 90-fold, 100-fold, 1000-fold, 5000-fold, 10,000-fold, 15,000-fold, 100,000-fold, 200,000-fold, 300,000-fold, 500,000-fold, 800,000-fold, or 1,000,000-fold more effective as compared to a non-multimeric construct. In aspects, a scaffold of the disclosure, such as a Cerberbody or a Hydrabody, is more effective at reducing or eliminating a target as compared to a monomeric or dimeric construct. In aspects, the effectiveness is at least about 1-fold, 10-fold, 20-fold, 30-fold, 40-fold, 50-fold, 60-fold, 70-fold, 80-fold, 90-fold, 100-fold, 1000-fold, 5000-fold, 10,000-fold, 15,000-fold, 100,000-fold, 200,000-fold, 300,000-fold, 500,000-fold, 800,000-fold, or 1,000,000-fold more effective as compared to a non-multimeric construct.

In aspects, a scaffold of the disclosure exhibits increased binding to a target as compared to non-multimeric scaffold or corresponding binding agent. In aspects, the binding is increased by at least about or at most about: 5%, 15%, 25%, 35%, 45%, 55%, 65%, 75%, 85%, 95%, 105%, 115%, 125%, 135%, 145%, 155%, 165%, 175%, 185%, 195%, 205%, 215%, 225%, 235%, 245%, 255%, 265%, 275%, 285%, 295%, 305%, 315%, 325%, 335%, 345%, 355%, 365%, 375%, 385%, 395%, 405%, 415%, 425%, 435%, 445%, 455%, 465%, 475%, 485%, 495%, or 500%.

In aspects, a multimeric construct, such as a Cerberbody or a Hydrabody, is more effective at reducing a symptom or disease in a subject in need thereof as compared to a monomeric or dimeric construct. In aspects, the effectiveness at reducing the symptom or disease is at least about 1-fold, 10-fold, 20-fold, 30-fold, 40-fold, 50-fold, 60-fold, 70-fold, 80-fold, 90-fold, 100-fold, 1000-fold, 5000-fold, 10,000-fold, 15,000-fold, 100,000-fold, 200,000-fold, 300,000-fold, 500,000-fold, 800,000-fold, or 1,000,000-fold more effective as compared to a non-multimeric construct.

In aspects, a multimeric construct, such as a Cerberbody or a Hydrabody, is more effective at neutralizing or reducing a pathogen or variant thereof (e.g., viral/bacterial/cancer variant) that has evaded a conventional therapy as compared to a monomeric or dimeric construct. In aspects, the effectiveness is at least about 1-fold, 10-fold, 20-fold, 30-fold, 40-fold, 50-fold, 60-fold, 70-fold, 80-fold, 90-fold, 100-fold, 1000-fold, 5000-fold, 10,000-fold, 15,000-fold, 100,000-fold, 200,000-fold, 300,000-fold, 500,000-fold, 800,000-fold, or 1,000,000-fold more effective as compared to the conventional therapy. In aspects, the effectiveness is increased by at least about or at most about: 5%, 15%, 25%, 35%, 45%, 55%, 65%, 75%, 85%, 95%, 105%, 115%, 125%, 135%, 145%, 155%, 165%, 175%, 185%, 195%, 205%, 215%, 225%, 235%, 245%, 255%, 265%, 275%, 285%, 295%, 305%, 315%, 325%, 335%, 345%, 355%, 365%, 375%, 385%, 395%, 405%, 415%, 425%, 435%, 445%, 455%, 465%, 475%, 485%, 495%, or 500%.

### Linkers

In aspects, provided are also linkers that can be utilized in the compositions and methods provided herein. Any linker can be utilized in the compositions of the disclosure for example to join one or more heterologous moieties of the disclosure. In aspects, a linker can also be utilized to join a Cerberbody and/or Hydrabody. In aspects, a linker comprises smAKAP.

In aspects, a heterologous moiety can be conjugated directly to a scaffold or via a linker. In aspects, two or more heterologous moieties can be conjugated directly or via a linker. Suitable linkers include, for example, cleavable and non-cleavable linkers. A cleavable linker is typically susceptible to cleavage under intracellular conditions. Suitable cleavable linkers include, for example, a peptide linker cleavable by an intracellular protease, such as lysosomal protease or an endosomal protease. In aspects, a linker can be a dipeptide linker, such as a valine-citrulline (val-cit) or a phenylalanine-lysine (phe-lys) linker. Other suitable linkers include linkers hydrolyzable at a pH of less than 5.5, such as a hydrazone linker. Additional suitable cleavable linkers include disulfide linkers.

In aspects, the linker is a rigid linker. In aspects, the linker is a flexible linker. In aspects, the linker attaches two or more VHH sequences. In aspects, the linker attaches one or more VHH sequences with another heterologous moiety of the disclosure. In aspects, the heterologous moiety is selected from the group consisting of: a chaperone protein, a targeting protein, a scaffold, an oligomerization domain, an enzyme, or a lysin.

In aspects, a linker comprises smAKAP peptide. In aspects, the smAKAP peptide can be modified. A modification can be done for example to increase soluble protein expression. In aspects, one or more residues in a linker can be mutated. In aspects, two Cys residues in an exemplary linker, e.g., smAKAP peptide, are mutated to serine. In aspects, a cleavable linker can also be utilized in compositions provided herein.

In aspects, a linker comprises at least about or at most about: 80%, 82%, 84%, 86%, 88%, 90%, 92%, 94%, 96%, 98%, 100% identity to a linker selected from the group consisting of: AEAAAKAS (a helix 1 linker; SEQ ID NO: 68), AEAAAKEAAAKAS (helix 2 linker; SEQ ID NO: 69), AEAAAKEAAAKEAAAKEAAAKAS (helix 4 linker; SEQ ID NO: 70), APAPSPAPSPAS (a PA5 linker; SEQ ID NO: 71), and a PA10 lin25.

In aspects, a linker comprises a mutant smAKAP that comprises an amino acid sequence with at least about 85%, 90%, 95%, 96%, 97%, 98%, 99%, or 100% identity to a sequence of SEQ ID NO: 5, 7, 15, 17, 19, 21, 72, 73, and 74. A linker can also comprise a WT smAKAP sequence of SEQ ID NO: 2.

In aspects, a linker can be of any length. In aspects, a linker is from about: 1-3, 2-6, 1-8, 3-10, 5-20, 3-15, 10-30, 15-35, 20-35, or 25-35 residues in length. In aspects, a linker is about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

### Spirulina

Spirulina is synonymous with “Arthrospira.” The genus Arthrospira includes 57 species of which 22 are currently taxonomically accepted. In aspects, a Spirulina species is selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In aspect, a Spirulina is A. platensis.

In aspects, the recombinant Spirulina is non-living. In aspects, the recombinant Spirulina is dried, spray dried, freeze-dried, or lyophilized.

Any appropriate means for transforming Spirulina may be used in the present disclosure. Exemplary methods for transforming Spirulina to express a heterologous protein are described in U.S. Pat. No. 10,131,870, which is incorporated by reference herein in its entirety.

In aspects, methods of making a Spirulina composition expressing a scaffold of the disclosure comprise introducing an expression vector having a nucleic acid sequence encoding the scaffold into a Spirulina cell. In aspects, the vector is not integrated into the Spirulina genome. In aspects, the vector is a high copy or a high expression vector. In aspects the nucleic acid sequence encoding the scaffold is under the control of a strong promoter. In aspects the nucleic acid sequence encoding the scaffold is under the control of a constitutive promoter. In aspects the nucleic acid sequence encoding the scaffold is under the control of an inducible promoter.

In aspects, methods of making a composition comprise introducing a vector (e.g. via homologous recombination) having homology arms and a nucleic acid sequence encoding a scaffold into a Spirulina cell.

In aspects, a vector having homology arms and a nucleic acid sequence encoding a scaffold can be introduced into Spirulina using electroporation. The electroporation is preferably carried out in the presence of an appropriate osmotic stabilizer.

Prior to introduction of the vector into Spirulina, Spirulina may be cultured in any suitable media for growth of cyanobacteria such as SOT medium. SOT medium includes NaHCO₃1.68 g, K2HPO₄ 50 mg, NaNO₃ 250 mg, K₂50₄ 100 mg, NaCl 100 mg, MgSO₄·7H₂O, 20 mg, CaCl₂·2H₂O 4 mg, FeSO₄·7H₂O 1 mg, Na₂EDTA·2H₂O 8 mg, A₅ solution 0.1 mL, and distilled water 99.9 mL. As solution includes H₃BO₃ 286 mg, MnSO₄·5H₂O) 217 mg, ZnSO₄·7H₂O 22.2 mg, CuSO₄·5H₂O 7.9 mg, Na₂MoO₄·2H₂O 2.1 mg, and distilled water 100 mL. Cultivation may occur with shaking (e.g., 100-300 rpm) at a temperature higher than room temperature (e.g. 25-37° C.) and under continuous illumination (e.g. 20-2,000, 50-500, or 100-200 μmol photon m⁻² s⁻¹). The growing cells may be harvested when the optical density at 750 nm reaches a predetermined threshold (e.g., OD₇₅₀ of 0.3-2.0, 0.5-1.0, or 0.6-0.8). A volume of the harvested cells may be concentrated by centrifugation then resuspended in a solution of pH balancer and salt. The pH balancer may be any suitable buffer that maintains viability of Spirulina while keeping pH of the media between 6 and 9 pH, between 6.5 and 8.5 pH, or between 7 and 8 pH. Suitable pH balancers include HEPES, HEPES-NaOH, sodium or potassium phosphate buffer, and TES. The salt solution may be NaCl at a concentration of between 50 mM and 500 mM, between 100 mM and 400 mM, or between 200 mM and 300 mM. In aspects, between 1-50 mL of 1-100 mM pH balance may be used to neutralize the pH.

Cells collected by centrifugation may be washed with an osmotic stabilizer and optionally a salt solution (e.g. 1-50 mL of 0.1-100 mM NaCl). Any amount of the culture may be concentrated by centrifugation. In aspects, between 5-500 mL of the culture may be centrifuged. The osmotic stabilizer may be any type of osmotic balancer that stabilizes cell integrity of Spirulina during electroporation. In aspects, the osmotic stabilizer may be a sugar (e.g. w/v 0.1-25%) such as glucose or sucrose. In aspects the osmotic stabilizer may be a simple polyol (e.g. v/v 1-25%) including glycerine, glycerin, or glycerol. In aspects the osmotic stabilizer may be a polyether including (e.g. w/v 0.1-20%) polyethylene glycol (PEG), poly(oxyethylene), or poly(ethylene oxide) (PEO). The PEG or PEO may have any molecular weight from 200 to 10,000, from 1000 to 6000, or from 2000 to 4000. In aspects the pH balancer or buffer may be used instead of or in addition to the osmotic stabilizer.

A vector having homology arms and a nucleic acid sequence encoding a scaffold can be introduced into Spirulina cells that are cultured and washed with an osmotic stabilizer as described above. Electroporation can be used to introduce the vector.

Electroporation may be performed in a 0.1-, 0.2- or 0.4-cm electroporation cuvette at between 0.6 and 10 kV/cm, between 2.5 and 6.5 kV/cm, or between 4.0 and 5.0 kV/cm; between 1 and 100 μF, between 30 and 70 μF, or between 45 and 55 μF; and between 10 and 500 mΩ, between 50 and 250 mΩ, or between 90 and 110 mΩ. In aspects, electroporation may be performed at 4.5 kV/cm, 50 μf, and 100 mΩ.

Following electroporation the cells may be grown in the presence of one or more antibiotics selected based on resistance conferred through successful transformation with the plasmid. Post-electroporation culturing may be performed at reduced illumination levels (e.g. 5-500, 10-100, or 30-60 μmol photon m⁻² s⁻¹). The culturing may also be performed with shaking (e.g. 100-300 rpm). The level of antibiotics in the media may be between 5 and 100 μg/mL. Post-electroporation culturing may be continued for 1-5 days or longer. Successful transformants identified by antibiotic resistance may be selected over a time course of 1 week to 1 month on plates or in 5-100 mL of SOT medium supplemented with 0.1-2.0 μg of appropriate antibiotics.

A vector used in the methods can be a plasmid, bacteriophage, or a viral vector into which a nucleic acid sequence encoding the at least one exogenous polypeptide, antigen, and/or antigen can be inserted or cloned. A vector may comprise one or more specific sequences that allow recombination into a particular, desired site of the Spirulina's chromosome. These specific sequences may be homologous to sequences present in the wild-type Spirulina. A vector system can comprise a single vector or plasmid, two or more vectors or plasmids, some of which increase the efficiency of targeted mutagenesis, or a transposition. The choice of the vector will typically depend on the compatibility of the vector with the Spirulina cell into which the vector is to be introduced. The vector can include a reporter gene, such as a green fluorescent protein (GFP), which can be either fused in frame to one or more of the encoded antigenic epitopes, or expressed separately. The vector can also include a positive selection marker such as an antibiotic resistance gene that can be used for selection of suitable transformants. The vector can also include a negative selection marker such as the type II thioesterase (tesA) gene or the Bacillus subtilis structural gene (sacB). Use of a reporter or marker allows for identification of those cells that have been successfully transformed with the vector.

In aspects, the vector includes one or two homology arms that are homologous to DNA sequences of the Spirulina genome that are adjacent to the targeted locus. The sequence of the homology arms can be partially or fully complementary to the regions of Spirulina genome adjacent to the targeted locus.

The homology arms can be of any length that allows for site-specific homologous recombination. A homology arm may be any length between about 2000 bp and 500 bp. For example, a homology arm may be about 2000 bp, about 1500 bp, about 1000 bp, or about 500 bp. In aspects having two homology arms, the homology arms may be the same or different length. Thus, each of the two homology arms may be any length between about 2000 bp and 500 bp. For example, each of the two homology arms may be about 2000 bp, about 1500 bp, about 1000 bp, or about 500 bp.

A portion of the vector adjacent to one homology arm or flanked by two homology arms modifies the targeted locus in the Spirulina genome by homologous recombination. The modification may change a length of the targeted locus including a deletion of nucleotides or addition of nucleotides. The addition or deletion may be of any length. The modification may also change a sequence of the nucleotides in the targeted locus without changing the length. The targeted locus may be any portion of the Spirulina genome including coding regions, non-coding regions, and regulatory sequences.

### Methods of Use

Compositions of the disclosure can be utilized in methods of treatment. In aspects, compositions of the present disclosure can be used to reduce the severity of a disease or disorder in a subject in need thereof. In aspects, compositions can be used to prevent a disease or disorder in a subject. In aspects, compositions can be used to prevent initiation of a disease or disorder in a subject. In aspects, compositions can be used to reduce the severity of a disease or disorder in a subject. In aspects, compositions can be used to prevent or delay recurrence of a disease in a subject. In aspects, compositions can be used to treat, prevent, or delay recurrence of a cancer in a subject. In aspects, compositions can be used to treat, prevent, or delay recurrence of a systemic pathogen. In aspects, compositions can be used to treat, prevent, or delay recurrence of a systemic parasite. In aspects, compositions can be used to treat, prevent, or delay recurrence of malaria. In aspects, compositions can be used to treat, prevent, or delay recurrence of a mucosal pathogen.

In aspects, a method of treatment comprises administering a disclosed composition for the treatment of an enteric pathogen. Exemplary enteric pathogens comprise: campylobacter disease, enterotoxigenic E. coli, norovirus, C. difficile, and SARS-COV-2.

In aspects, a composition of the disclosure is utilized to treat a disease or condition associated with, or derived from, or treats or prevents infection by any microorganism, including, but not limited to, E. coli, Enterotoxigenic E. coli (ETEC), anthrax, EHEC, EAEC, Shigella, Mycobacterium, Streptococcus, Staphylococcus, Shigella, Campylobacter, Salmonella, Clostridium, Corynebacterium, Pseudomonas, Neisseria, Listeria, Vibrio, Bordetella. Legionella, bacteriophage, RNA bacteriophage (e.g. MS2, AP205, PP7 and QB), Helicobacter pylori, Infectious Hematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, White Spot Syndrome Virus, a coronavirus, SARS, MERS, SARS-COV-2, Aspergillus, Candida, Blastomyces, Coccidioides, Cryptococcus, Histoplasma, Plasmodium, P. falciparum, P. malariae, P. ovale, P. vivax, Trypanosoma, Toxoplasma, Giardia, Leishmania Cryptosporidium, helminthic parasites: Trichuris spp., Enterobius spp., Ascaris spp., Ancylostoma spp. and Necatro spp., Strongyloides spp., Dracunculus spp., Onchocerca spp. and Wuchereria spp., Taenia spp., Echinococcus spp., and Diphyllobothrium spp., Fasciola spp., and Schistosoma spp. or a combination thereof.

In aspects, compositions described herein can be used to treat and/or reduce the severity of an infection caused by a bacterium including, but not limited to: Mycobacterium, Streptococcus, Staphylococcus, Shigella, Campylobacter, Salmonella, Clostridium, Corynebacterium, Pseudomonas, Neisseria, Listeria, Vibrio, Bordetella, and Legionella.

In aspects, compositions described herein can be used to treat or reduce the severity of an infection caused by a parasite including, but not limited to, Plasmodium, Trypanosoma, Toxoplasma, Giardia, and Leishmania, Cryptosporidium, helminthic parasites: Trichuris spp. (whipworms), Enterobius spp. (pinworms), Ascaris spp. (roundworms), Ancylostoma spp. and Necatro spp. (hookworms), Strongyloides spp. (threadworms), Dracunculus spp. (Guinea worms), Onchocerca spp. and Wuchereria spp. (filarial worms), Taenia spp., Echinococcus spp., and Diphyllobothrium spp. (human and animal cestodes), Fasciola spp. (liver flukes) and Schistosoma spp. (blood flukes).

In aspects, compositions described herein can be used to treat or reduce the severity of an infection caused by Plasmodium. In aspects, compositions of the present disclosure can be used to induce an immune response to and/or reduce the severity of an infection caused by a Plasmodium selected from the group consisting of: P. falciparum, P. malariae, P. ovale and P. vivax.

In aspects, compositions described herein can be used to treat or reduce the severity of an infection caused by a fungus including but not limited to Aspergillus, Candida, Blastomyces, Coccidioides, Cryptococcus, and Histoplasma. In aspects, compositions can be used to induce an immune response to and/or reduce the severity of a Candida albicans or a Candida auris infection.

In aspects, compositions described herein can be used to treat or reduce breast cancer cell, colon cancer cell, brain cancer cell, pancreatic cancer cell, lung cancer cell, cervical cancer cell, uterine cancer cell, prostate cancer cell, ovarian cancer cell, melanoma cancer cell, lymphoma cancer cell, myeloma cancer cell, and leukemic cancer cell.

In aspects, compositions described herein can be used to treat or reduce symptoms of autoimmune disease including but not limited to ulcerative colitis, rheumatoid arthritis, systemic lupus erythematosus (SLE), celiac disease, inflammatory bowel disease, Hashimoto's disease, Addison's disease, Grave's disease, type I diabetes, autoimmune thrombocytopeni purpura (ATP), idiopathic pulmonary fibrosis, idiopathic thrombocytopenia purpura (ITP), Crohn's disease, multiple sclerosis, and myasthenia gravis.

Other GI targets such as inflammatory and metabolic diseases, and manipulation of the microbiome, are also contemplated. In aspects, the compositions described herein can alter the abundance of microorganisms present in the microbiome. In aspects, the compositions described herein can alter the abundance of bacteria present in the microbiome. In aspects, the compositions disclosed herein can alter the abundance of Firmicutes, Bacteroidetes, Proteobacteria, and Actinobacteria. In aspects, the compositions described herein can decrease the abundance of pro-inflammatory bacteria in the microbiome. In aspects, the compositions described herein can be used to decrease the abundance of bacteria comprising Bacteroides, Proteobacteria, Enterobacteriaceae, Porphyromonas, Clostridiales, Pasteurellaceae, Veillonellaceae, Neisseriaceae, Clostridia, Proetobacteria, Ruminococcus, Dorea, Actinomyces, and Fusobacteriaceae. In aspects, compositions described herein can be used to decrease the abundance of bacteria comprising Escherichia coli, Shigella, Rhodococcus, Veillonella, Stenotrophomonas maltophilia, Prevotellaceae, Clostridium difficile, Clostridium ramosum, Klebsiella pneumoniae, Proteus mirabilis, Staphylococcus aureus, Ruminococcus torques, Ruminococcus gnavus and Helicobacter hepaticus. In aspects, the compositions described herein can be used to increase the abundance of anti-inflammatory bacteria in the microbiome. In aspects, the compositions described herein can be used to increase butyrate producing bacteria. In aspects, compositions described herein can be used to increase bacteria comprising Firmicutes, Lactobacillus, Rumenococcaceae, Eubacterium, Bifidobacterium, Faecalibacterium, Roseburia, Coprococcus eutactus, Roseburia, Akkermansia and Clostridiales. In aspects, the compositions described herein can be used to increase bacteria comprising Faecalibacterium prausnitzii, Bifidobacterium longum, Eubacterium rectale, Roseburia intestinalis, and Akkemansia mucinophila.

### Pharmaceutical Compositions

In aspects, a composition of the disclosure is formulated as a pharmaceutical composition. For example, a scaffold can be comprised within a pharmaceutical composition. Pharmaceutical compositions of the disclosure can be administered to a subject in need thereof. Administration can be conducted in any way. In aspects, administration is oral, intravenous, intradermal, via the airway, intranasal, and combinations thereof.

In aspects, a pharmaceutical composition is administered orally. As used herein, the terms “oral composition” or “orally delivered composition” comprise compositions administered or delivered to the gastrointestinal tract (e.g., orally, compositions administered to the stomach via a feeding tube, etc.). Any appropriate area of the gastrointestinal tract may be targeted by the compositions of the present disclosure.

In aspects, the compositions of the present disclosure are administered via the airway. In aspects, the compositions of the present disclosure are administered by inhalation. In aspects, the compositions of the present disclosure are administered intranasally. In aspects, the compositions of the present disclosure are administered by a nebulizer, an inhaler, or a mist. In aspects, the compositions of the present disclosure are lyophilized and delivered as a powder, or a powder resuspended in a liquid. In aspects, the compositions of the present disclosure are formulated for administration by a nebulizer, an inhaler, a dry powder inhalation device, or a mist.

In aspects, the pharmaceutical composition comprises an adjuvant. Most adjuvants contain a substance designed to protect a protein or fragment thereof from rapid catabolism such as aluminum hydroxide or mineral oil, and a stimulator of immune responses, such as Bordetella pertussis or Mycobacterium tuberculosis derived proteins. In aspects, adjuvants comprise, but are not limited to, toll-like receptor (TLR) agonists, monophosphoryl lipid A (MPL), synthetic lipid A, lipid A mimetics or analogs, aluminum salts, cytokines, saponins, muramyl dipeptide (MDP) derivatives, CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, montanide and liposomes. In aspects, the adjuvant is a montanide adjuvant.

In aspects, the composition comprises at least one adjuvant. In aspects, the adjuvant is present in solution. In aspects, the adjuvant is present at a concentration of about 1 μg/mL to about 85 mg/mL (e.g. 1 μg/mL, 50 μg/mL, 100 μg/mL, 150 μg/mL, 200 μg/mL, 250 μg/mL, 300 μg/mL, 350 μg/mL, 400 μg/mL, 450 μg/mL, 500 μg/mL, 550 μg/mL, 600 μg/mL, 650 μg/mL, 700 μg/mL, 750 μg/mL, 800 μg/mL, 850 μg/mL, 900 μg/mL, 950 μg/mL, 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, or 85 mg/mL, including all values and ranges therein).

In aspects, the composition survives in the gastrointestinal tract or a simulated stomach environment. In aspects, the composition survives in the gastrointestinal tract or a simulated stomach environment for at least 5 minutes. In aspects, the composition survives in the gastrointestinal tract or a simulated stomach environment overnight.

In aspects, the composition survives in the nasal cavity. In aspects, the composition survives in the upper respiratory tract. In aspects, the composition survives in the airway. In aspects, the composition survives in the nasal cavity, upper respiratory tract and/or the airway for at least 5 minutes. In aspects, the composition survives in the nasal cavity, upper respiratory tract and/or the airway overnight.

The compositions of the present disclosure may be administered daily, weekly, biweekly, every other week, monthly, etc. In aspects, the compositions of the present disclosure are administered to a subject for about 1 day to about 1 year. In aspects, the compositions of the present disclosure are administered to a subject for about 1 day, 2 days, 3 days, 4 days, 5 days, 6 days, 7 days, 8 days, 9 days, 10 days, one week, two weeks, three weeks, four weeks, five weeks, six weeks, one month, two months, three months, four months, five months or more. In aspects, the compositions of the present disclosure are administered on consecutive days. In aspects, the compositions of the present disclosure are administered on non-consecutive days. In aspects, the compositions of the present disclosure are administered once a day. In aspects, the compositions of the present disclosure are administered multiple times a day. In aspects, the compositions of the present disclosure are administered twice a day, three times a day, four times a day, or more. In aspects, the compositions of the present disclosure are administered continuously (e.g. via a feeding tube). In aspects, the compositions of the present disclosure are administered with meals. In aspects, the compositions of the present disclosure are administered when the subject is in a fasting state.

In aspects, compositions of the present disclosure can comprise one or more pharmaceutically acceptable excipients. Pharmaceutically acceptable carriers include but are not limited to saline, buffered saline, dextrose, water, glycerol, sterile isotonic aqueous buffer, and combinations thereof. In aspects, a pharmaceutically acceptable excipient is sodium bicarbonate.

In aspects, the composition further comprises an excipient. In aspects, the composition comprises at least one excipient. In aspects, the composition comprises at least two or more excipients. In aspects, the composition comprises three or more excipients. In aspects, the composition comprises four or more excipients. In aspects, the composition comprises five or more excipients. In aspects, the composition comprises six or more excipients. In aspects, the composition comprises seven or more excipients. Exemplary excipients comprises but are not limited to stabilizers, buffers, surfactants, bulking agents, sugars and salts.

In aspects, the excipient is selected from a group comprising of stabilizers, buffers, surfactants, bulking agents, sugars and salts. Exemplary stabilizers comprise but are not limited to lactose, gelatin, sucrose, sorbitol, human serum albumin, aluminum salts, monosodium glutamate, sodium chloride, mannitol, L-histidine and dextran. Exemplary buffers comprise but not limited to hydrogen chloride, general buffers, pH counter buffers, phosphate buffered saline (PBS), phosphate buffer, acetate buffer, tris buffer, HEPES buffer, glycine buffer, citrate buffer, histidine buffer. Exemplary surfactants comprise but not limited to polyethylene glycol tert-octylphenyl ether, polysorbate-80, sorbitan monooleate, sodium dodecyl sulfate, poloxamer 188, cetyltrimethylammonium bromide, and polysorbate-20. Exemplary bulking agents comprise but are not limited to sucrose, lactose, mannitol, sodium chloride, gelatin, aluminum salts, and hydrolyzed gelatin. Exemplary sugars comprise but are not limited to sucrose, lactose, mannitol, dextrose, fructose, and trehalose. Exemplary salts comprise but are not limited to sodium chloride, potassium chloride, sodium phosphate, potassium phosphate, potassium citrate, aluminum salts, calcium chloride, magnesium chloride, sodium sulfate, ammonium sulfate, and sodium citrate. In aspects, the excipient further comprises histidine, arginine and glycine.

In aspects, the excipient is present in solution. In aspects, the excipients is present at a concentration from about 1 μg/mL to about 1 g/mL (e.g. 1 μg/mL, 50 μg/mL, 100 μg/mL, 150 μg/mL, 200 μg/mL, 250 μg/mL, 300 μg/mL, 350 μg/mL, 400 μg/mL, 450 μg/mL, 500 μg/mL, 550 μg/mL, 600 μg/mL, 650 μg/mL, 700 μg/mL, 750 μg/mL, 800 μg/mL, 850 μg/mL, 900 μg/mL, 950 μg/mL, 1,000 μg/mL, 50 mg/mL, 100 mg/mL, 150 mg/mL, 200 mg/mL, 250 mg/mL, 300 mg/mL, 350 mg/mL, 400 mg/mL, 450 mg/mL, 500 mg/mL, 550 mg/mL, 600 mg/mL, 650 mg/mL, 700 mg/mL, 750 mg/mL, 800 mg/mL, 850 mg/mL, 900 mg/mL, 950 mg/mL, 1 g/mL, 1.5 g/mL, 2 g/mL, or up to 2.5 g/mL including all values and ranges therein).

In aspects, an excipient is from about 0.1% to about 0.5% of a composition. An excipient can be from about 0.1%, 0.2%, 0.3%, 0.4%, or up to about 0.5% of a composition.

### Kits

Disclosed herein are also kits comprising disclosed compositions. Disclosed herein can also be kits for the treatment or prevention of a cancer, pathogen infection, or immune disorder. aspects, a kit can include a therapeutic or prophylactic composition containing an effective amount of a composition comprising a recombinant Spirulina (e.g., transformed with a scaffold) in unit dosage form. In aspects, a kit comprises a sterile container which can contain a therapeutic composition of Spirulina; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In aspects, transformed Spirulina, can be provided together with instructions for administering the Spirulina to a subject having or at risk of developing a cancer, pathogen infection, or immune disorder.

### Numbered Embodiments

Notwithstanding the appended claims, the following numbered embodiments also form part of the instant disclosure.

### Embodiment Set 1

1. A recombinant Spirulina, that expresses a mutated small-membrane A-kinase anchoring protein (smAKAP) peptide sequence.

2. The recombinant Spirulina of embodiment 1, wherein the Spirulina comprises at least 2, 3, 4, 5, 6, or 7 mutated residues in the mutant smAKAP peptide sequence as compared to a WT smAKAP peptide sequence.

3. The recombinant Spirulina of embodiment 1, wherein 1 residue in the smAKAP peptide sequence is mutated as compared to a WT smAKAP peptide sequence.

4. The recombinant Spirulina of any one of embodiments 2-3, wherein 2 residues in the smAKAP peptide sequence are mutated as compared to a WT smAKAP peptide sequence.

5. The recombinant Spirulina of any one of embodiments 2-4, wherein 3 residues in the smAKAP peptide sequence are mutated as compared to a WT smAKAP peptide sequence.

6. The recombinant Spirulina of any one of embodiments 1-5, wherein the mutated smAKAP peptide exhibits resistance to protease cleavage as determined by reduced detection of cleavage products when exposed to a solvent comprising a protease for one hour.

7. The recombinant Spirulina of embodiment 6, wherein at most about 5%, 10%, 20%, 30%, 40%, 50%, or 60% of the smAKAP peptide sequence is cleaved.

8. The recombinant Spirulina of any one of embodiments 1-7, wherein the mutation is of a hydrophobic residue.

9. The recombinant Spirulina of any one of embodiments 1-8, wherein when the recombinant Spirulina is submerged in a solvent, the mutated residue is exposed to the solvent.

10. The recombinant Spirulina of any one of embodiments 1-9, wherein the mutated residue is selected from the group consisting of: C16S, C24S, E5D, Y6H, W22S, C24G, L4E, R9E, L4I, L10I, L19I, and combination thereof of SEQ ID NO: 2.

11. The recombinant Spirulina of embodiment 10, wherein the mutated residue is C16S and C24S as compared to SEQ ID NO: 2.

12. The recombinant Spirulina of embodiment 10, wherein the mutated residue is E5D, Y6H, C16S, W22S, and C24G as compared to SEQ ID NO: 2.

13. The recombinant Spirulina of embodiment 10, wherein the mutated residue is LAE, E5D, Y6H, R9E, C16S, W22S, and C24G as compared to SEQ ID NO: 2.

14. The recombinant Spirulina of embodiment 10, wherein the mutated residue is LAE, C16S, W22S, and C24G as compared to SEQ ID NO: 2.

15. The recombinant Spirulina of embodiment 10, wherein the mutated residue is R9E, C16S, and C24G as compared to SEQ ID NO: 2.

16. The recombinant Spirulina of embodiment 10, wherein the mutated residue is R9E, C16S, W22S, and C24G as compared to SEQ ID NO: 2.

17. The recombinant Spirulina of any one of embodiments 1-16, wherein the mutant smAKAP peptide sequence is linked to a heterologous moiety in a monomeric configuration.

18. The recombinant Spirulina of embodiment 17, wherein the mutant smAKAP peptide sequence is linked to a second heterologous moiety.

19. The recombinant Spirulina of embodiment 18, wherein the heterologous moiety and the second heterologous moiety are the same.

20. The recombinant Spirulina of any one of embodiments 17-18, wherein the heterologous moiety and the second heterologous moiety are the different.

21. The recombinant Spirulina of any one of embodiments 1-20, wherein the recombinant Spirulina expresses another exogenous polypeptide sequence.

22. The recombinant Spirulina of embodiment 21, wherein the exogenous polypeptide sequence is selected from the group consisting of: oligomerization domain of C4b-binding protein (C4BP), cholera toxin b subunit, oligomerization domains of extracellular matrix proteins, TRX, 5HVZ, cTRP, SP651, SP737, and 4B0F.

23. The recombinant Spirulina of embodiment 22, wherein the exogenous polypeptide sequence is 5HVZ.

24. The recombinant Spirulina of embodiment 23, wherein the 5HVZ is linked to a third heterologous moiety

25. The recombinant Spirulina of embodiment 24, wherein the third heterologous moiety is in a homodimeric configuration.

26. The recombinant Spirulina of any one of embodiments 17-25, wherein the heterologous moiety, the second heterologous moiety, and the third heterologous moieties are the same.

27. The recombinant Spirulina of any one of embodiments 17-25, wherein the heterologous moiety, the second heterologous moiety, and the third heterologous moieties are the different.

28. The recombinant Spirulina of any one of embodiments 17-27, wherein the heterologous moiety is a binding agent.

29. The recombinant Spirulina of embodiment 28, wherein the binding agent is selected from the group consisting of: fab′, F(ab′) 2, fv, domain antibody (dAb), complementarity Determining Region (CDR) fragment, CDR-grafted antibody, single chain antibodies (scFv), single chain antibody fragment, chimeric antibody, diabody, triabody, tetrabody, minibody, linear antibody, intrabody, nanobody (single domain antibody), small Modular Immunopharmaceuticals (SMIPs), antigen-binding domain immunoglobulin fusion protein, and VHH.

30. The recombinant Spirulina of embodiment 29, wherein the binding agent is the VHH.

31. The recombinant Spirulina of embodiment 30, wherein the VHH binds a pathogen.

32. The recombinant Spirulina of embodiment 30, wherein the VHH binds a cancer cell.

33. The recombinant Spirulina of embodiment 30, wherein the VHH binds a human cell.

34. The recombinant Spirulina of embodiment 31, wherein the VHH binds a pathogen selected from the group consisting of: bacteria, fungi, and virus.

35. The recombinant Spirulina of embodiment 34, wherein the pathogen is selected from the group consisting of: E. coli, Enterotoxigenic E. coli (ETEC), anthrax, EHEC, EAEC, Shigella, Mycobacterium, Streptococcus, Staphylococcus, Shigella, Campylobacter, Salmonella, Clostridium, Corynebacterium, Pseudomonas, Neisseria, Listeria, Vibrio, Bordetella, Legionella, bacteriophage, RNA bacteriophage (e.g. MS2, AP205, PP7 and QB), Helicobacter pylori, Infectious Hematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, White Spot Syndrome Virus, a coronavirus, SARS, MERS, SARS-COV-2, Aspergillus, Candida, Blastomyces, Coccidioides, Cryptococcus, Histoplasma, Plasmodium, P. falciparum, P. malariae, P. ovale, P. vivax, Trypanosoma, Toxoplasma, Giardia, Leishmania Cryptosporidium, helminthic parasites: Trichuris spp., Enterobius spp., Ascaris spp., Ancylostoma spp. and Necatro spp., Strongyloides spp., Dracunculus spp., Onchocerca spp. Wuchereria spp., Taenia spp., Echinococcus spp., and Diphyllobothrium spp., Fasciola spp., and Schistosoma spp.

36. The recombinant Spirulina of embodiment 34, wherein the pathogen is bacteria and the bacteria is selected from the group consisting of: Mycobacterium, Streptococcus, Staphylococcus, Shigella, Campylobacter, Salmonella, Clostridium, Corynebacterium, Pseudomonas, Neisseria, Listeria, Vibrio, Bordetella, and Legionella.

37. The recombinant Spirulina of embodiment 36, wherein the bacteria is Campylobacter.

38. The recombinant Spirulina of embodiment 36, wherein the bacteria is Clostridium.

39. The recombinant Spirulina of any one of embodiments 35-38, wherein the VHH comprises a sequence with at least 85% identity to a sequence of SEQ ID NO: 25-67.

40. The recombinant Spirulina of any one of embodiments 35-38, wherein the VHH comprises a sequence SEQ ID NO: 25-67.

41. The recombinant Spirulina of any one of embodiments 23-40, wherein the recombinant Spirulina expresses a scaffold having at least 85%, 90%, 95%, 96%, 97%, 98%, 99%, or 100% identity to a sequence of SEQ ID NO: 6, 14, 16, 18, 20, 22, 23, and 24.

42. A recombinant Spirulina, that expresses a mutated small-membrane A-kinase anchoring protein (smAKAP) peptide sequence, wherein the mutation comprises a substitution of a residue of SEQ ID NO: 2.

43. A recombinant Spirulina, that expresses a mutated small-membrane A-kinase anchoring protein (smAKAP) peptide sequence, wherein the mutation is selected from the group consisting of: C16S, C24S, E5D, Y6H, W22S, C24G, L4E, R9E, LAI, L10I, L19I, and combination thereof of SEQ ID NO: 2.

44. The recombinant Spirulina of any one of embodiments 42-43, wherein the smAKAP is linked to one or more VHH antibodies at a terminal end.

45. The recombinant Spirulina of embodiment 44, wherein the smAKAP is linked to two VHH antibodies, wherein a first VHH is at a C-terminus and the second VHH is at an N-terminus.

46. The recombinant Spirulina of any one of embodiments 42-45, wherein the recombinant Spirulina expresses a 5HVZ.

47. The recombinant Spirulina of embodiment 46, wherein the 5HVZ is linked to two VHH antibodies in a homodimeric configuration.

48. A polynucleotide sequence comprising a mutated small-membrane A-kinase anchoring protein (smAKAP) sequence, wherein the mutation a single residue substitution as compared to a WT smAKAP sequence.

49. A vector comprising the polynucleotide sequence of embodiment 48.

50. A method of making a recombinant Spirulina comprising contacting a Spirulina cell with the vector of embodiment 49.

51. A pharmaceutical composition comprising the recombinant Spirulina of any one of embodiments 1-47, and an excipient.

52. A kit comprising: the recombinant Spirulina of any one of embodiments 1-47, the polynucleotide sequence of embodiment 48, the vector of embodiment 49, or the pharmaceutical composition of embodiment 51, and instructions for use thereof.

53. A method of treatment comprising administering the pharmaceutical composition of embodiment 51 to a subject in need thereof, wherein the subject comprises a bacterial or viral infection.

54. A vector comprising a nucleotide sequence encoding a polypeptide of SEQ ID NO: 5-7, or 14-24.

55. A recombinant Spirulina comprising the vector of embodiment 54.

### Embodiment Set 2

1. A recombinant Spirulina, that expresses a mutated small-membrane A-kinase anchoring protein (smAKAP) peptide sequence.

2. The recombinant Spirulina of embodiment 1, wherein the Spirulina comprises at least 2, 3, 4, 5, 6, or 7 mutated residues in the mutant smAKAP peptide sequence as compared to a WT smAKAP peptide sequence.

3. The recombinant Spirulina of embodiment 1, wherein 1 residue in the smAKAP peptide sequence is mutated as compared to a WT smAKAP peptide sequence.

4. The recombinant Spirulina of any one of embodiments 2-3, wherein 2 residues in the smAKAP peptide sequence are mutated as compared to a WT smAKAP peptide sequence.

5. The recombinant Spirulina of any one of embodiments 2-4, wherein 3 residues in the smAKAP peptide sequence are mutated as compared to a WT smAKAP peptide sequence.

6. The recombinant Spirulina of any one of embodiments 1-5, wherein the mutated smAKAP peptide exhibits resistance to protease cleavage as determined by reduced detection of cleavage products when exposed to a solvent comprising a protease for one hour.

7. The recombinant Spirulina of embodiment 6, wherein at most about 5%, 10%, 20%, 30%, 40%, 50%, or 60% of the smAKAP peptide sequence is cleaved.

8. The recombinant Spirulina of any one of embodiments 1-7, wherein the mutation is of a hydrophobic residue.

9. The recombinant Spirulina of any one of embodiments 1-8, wherein when the recombinant Spirulina is submerged in a solvent, the mutated residue is exposed to the solvent.

10. The recombinant Spirulina of any one of embodiments 1-9, wherein the mutated residue is selected from the group consisting of: C16S, C24S, E5D, Y6H, W22S, C24G, L4E, R9E, L4I, L10I, L19I, and combination thereof of SEQ ID NO: 2.

11. The recombinant Spirulina of embodiment 10, wherein the mutated residue is C16S and C24S as compared to SEQ ID NO: 2.

12. The recombinant Spirulina of embodiment 10, wherein the mutated residue is ESD, Y6H, C16S, W22S, and C24G as compared to SEQ ID NO: 2.

13. The recombinant Spirulina of embodiment 10, wherein the mutated residue is LAE, E5D, Y6H, R9E, C16S, W22S, and C24G as compared to SEQ ID NO: 2.

14. The recombinant Spirulina of embodiment 10, wherein the mutated residue is LAE, C16S, W22S, and C24G as compared to SEQ ID NO: 2.

15. The recombinant Spirulina of embodiment 10, wherein the mutated residue is R9E, C16S, and C24G as compared to SEQ ID NO: 2.

16. The recombinant Spirulina of embodiment 10, wherein the mutated residue is R9E, C16S, W22S, and C24G as compared to SEQ ID NO: 2.

17. The recombinant Spirulina of embodiment 10, wherein the mutated residue is LAI, R9E, C16S, C24G as compared to SEQ ID NO: 2.

18. The recombinant Spirulina of any one of embodiments 1-17, wherein the mutant smAKAP peptide sequence is linked to a heterologous moiety in a monomeric configuration.

19. The recombinant Spirulina of embodiment 18, wherein the mutant smAKAP peptide sequence is linked to a second heterologous moiety.

20. The recombinant Spirulina of embodiment 19, wherein the heterologous moiety and the second heterologous moiety are the same.

21. The recombinant Spirulina of any one of embodiments 18-19, wherein the heterologous moiety and the second heterologous moiety are the different.

22. The recombinant Spirulina of any one of embodiments 1-21, wherein the recombinant Spirulina expresses another exogenous polypeptide sequence.

23. The recombinant Spirulina of embodiment 22, wherein the exogenous polypeptide sequence is selected from the group consisting of: oligomerization domain of C4b-binding protein (C4BP), cholera toxin b subunit, oligomerization domains of extracellular matrix proteins, TRX, 5HVZ, cTRP, SP651, SP737, and 4B0F.

24. The recombinant Spirulina of embodiment 23, wherein the exogenous polypeptide sequence is 5HVZ.

25. The recombinant Spirulina of embodiment 24, wherein the 5HVZ is linked to a third heterologous moiety

26. The recombinant Spirulina of embodiment 25, wherein the third heterologous moiety is in a dimeric configuration.

27. The recombinant Spirulina of any one of embodiments 18-26, wherein the heterologous moiety, the second heterologous moiety, and the third heterologous moieties are the same.

28. The recombinant Spirulina of any one of embodiments 18-26, wherein the heterologous moiety, the second heterologous moiety, and the third heterologous moieties are the different.

29. The recombinant Spirulina of any one of embodiments 18-28, wherein the heterologous moiety is a binding agent.

30. The recombinant Spirulina of embodiment 29, wherein the binding agent is selected from the group consisting of: fab′, F(ab′) 2, fv, domain antibody (dAb), complementarity Determining Region (CDR) fragment, CDR-grafted antibody, single chain antibodies (scFv), single chain antibody fragment, chimeric antibody, diabody, triabody, tetrabody, minibody, linear antibody, intrabody, nanobody (single domain antibody), small Modular Immunopharmaceuticals (SMIPs), antigen-binding domain immunoglobulin fusion protein, and VHH.

31. The recombinant Spirulina of embodiment 30, wherein the binding agent is the VHH.

32. The recombinant Spirulina of embodiment 31, wherein the VHH binds a pathogen.

33. The recombinant Spirulina of embodiment 31, wherein the VHH binds a cancer cell.

34. The recombinant Spirulina of embodiment 31, wherein the VHH binds a human cell.

35. The recombinant Spirulina of embodiment 32, wherein the VHH binds a pathogen selected from the group consisting of: bacteria, fungi, and virus.

36. The recombinant Spirulina of embodiment 35, wherein the pathogen is selected from the group consisting of: E. coli, Enterotoxigenic E. coli (ETEC), anthrax, EHEC, EAEC, Shigella, Mycobacterium, Streptococcus, Staphylococcus, Shigella, Campylobacter, Salmonella, Clostridium, Corynebacterium, Pseudomonas, Neisseria, Listeria, Vibrio, Bordetella, Legionella, bacteriophage, RNA bacteriophage (e.g. MS2, AP205, PP7 and QB), Helicobacter pylori, Infectious Hematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, White Spot Syndrome Virus, a coronavirus, SARS, MERS, SARS-COV-2, Aspergillus, Candida, Blastomyces, Coccidioides, Cryptococcus, Histoplasma, Plasmodium, P. falciparum, P. malariae, P. ovale, P. vivax, Trypanosoma, Toxoplasma, Giardia, Leishmania Cryptosporidium, helminthic parasites: Trichuris spp., Enterobius spp., Ascaris spp., Ancylostoma spp. and Necatro spp., Strongyloides spp., Dracunculus spp., Onchocerca spp. Wuchereria spp., Taenia spp., Echinococcus spp., and Diphyllobothrium spp., Fasciola spp., and Schistosoma spp.

37. The recombinant Spirulina of embodiment 35, wherein the pathogen is bacteria and the bacteria is selected from the group consisting of: Mycobacterium, Streptococcus, Staphylococcus, Shigella, Campylobacter, Salmonella, Clostridium, Corynebacterium, Pseudomonas, Neisseria, Listeria, Vibrio, Bordetella, and Legionella.

38. The recombinant Spirulina of embodiment 37, wherein the bacteria is Campylobacter.

39. The recombinant Spirulina of embodiment 37, wherein the bacteria is Clostridium.

40. The recombinant Spirulina of any one of embodiments 36-39, wherein the VHH comprises a sequence with at least 85% identity to a sequence of SEQ ID NO: 25-67.

41. The recombinant Spirulina of any one of embodiments 36-39, wherein the VHH comprises a sequence SEQ ID NO: 25-67.

42. The recombinant Spirulina of any one of embodiments 24-41, wherein the recombinant Spirulina expresses a scaffold having at least 85%, 90%, 95%, 96%, 97%, 98%, 99%, or 100% identity to a sequence of SEQ ID NO: 4, 6, 14, 16, 18, 20, 22, 23, 24, and 76.

43. A recombinant Spirulina, that expresses a mutated small-membrane A-kinase anchoring protein (smAKAP) peptide sequence, wherein the mutation comprises a substitution of a residue of SEQ ID NO: 2.

44. A recombinant Spirulina, that expresses a mutated small-membrane A-kinase anchoring protein (smAKAP) peptide sequence, wherein the mutation is selected from the group consisting of: C16S, C24S, E5D, Y6H, W22S, C24G, L4E, R9E, LAI, L10I, L19I, and combination thereof of SEQ ID NO: 2.

45. The recombinant Spirulina of any one of embodiments 43-44, wherein the smAKAP is linked to one or more VHH antibodies at a terminal end.

46. The recombinant Spirulina of embodiment 45, wherein the smAKAP is linked to two VHH antibodies, wherein a first VHH is at a C-terminus and the second VHH is at an N-terminus.

47. The recombinant Spirulina of any one of embodiments 43-46, wherein the recombinant Spirulina expresses a 5HVZ.

48. The recombinant Spirulina of embodiment 47, wherein the 5HVZ is linked to two VHH antibodies in a homodimeric configuration.

49. A polynucleotide sequence comprising a mutated small-membrane A-kinase anchoring protein (smAKAP) sequence, wherein the mutation comprises one or more residue substitutions as compared to a WT smAKAP sequence.

50. A vector comprising the polynucleotide sequence of embodiment 49.

51. A method of making a recombinant Spirulina comprising contacting a Spirulina cell with the vector of embodiment 50.

52. A pharmaceutical composition comprising the recombinant Spirulina of any one of embodiments 1-48, and an excipient.

53. A kit comprising: the recombinant Spirulina of any one of embodiments 1-48, the polynucleotide sequence of embodiment 49, the vector of embodiment 50, or the pharmaceutical composition of embodiment 52, and instructions for use thereof.

54. A method of treatment comprising administering the pharmaceutical composition of embodiment 52 to a subject in need thereof, wherein the subject comprises a bacterial or viral infection.

55. A vector comprising a nucleotide sequence encoding a polypeptide of SEQ ID NO: 2-7, 14-24, 74, and/or 76.

56. The vector of embodiment 55, wherein the vector comprises from about 80%, 85%, 90%, 95% 96% 97%, 98%, 99%, or 100% identity to SEQ ID NO: 75.

56. A recombinant Spirulina comprising the vector of any one of embodiments 55-56.

### EXAMPLES

### Example 1—Evaluation of Protease Sensitivity

### Study Objectives

To evaluate protease sensitivity of exemplary scaffold constructs, the hydrabody complex, pp 2130 (complex of pp 1113 and pp 1895) was compared against the protease sensitivity of constructs: PP917 2xS3b-C8, pp 1895 2xS3b-C8, and pp 1895 2xRN-29.

Briefly, to Assess in vitro protease stability of clones, purified protein samples were prepared at 0.2 mg/mL in protease digestion buffer (20 mM Bis-Tris, 150 mM NaCl, 3 mM CaCl2 pH 6.0). The protease enzymes trypsin and chymotrypsin were diluted from 1 mg/mL stalk concentration to 0.2 mg/mL in protease digest buffer. Both the protein and enzyme were diluted 1:10 in protease digestion buffer. The digestion reactions were conducted by mixing 30 μL of diluted protein and 30 μL of diluted protease. The reactions were mixed by centrifugation for 30 seconds at high speed and incubated at 37° C. for 60 minutes with shaking at 900 rpm in an Eppendorf ThermoMixer. Reactions were quenched with an equal volume of stop buffer containing 2 mM PMSF and 2× Pierce protease inhibitor minitablet in PBS. The final concentration of each VHH homodimer after protease neutralization was 0.1 mg/mL. Samples were kept on ice for ELISA assay.

Digested protein samples were analyzed for binding activity via ELISA. High-binding ELISA plates were coated with 100 μl/well of 1 μg/mL SARS-COV-2 RBD in CBC binding buffer and incubated overnight at 4° C. Plates were washed 3× with PBS supplemented with 0.05% Tween-20 (PBS-tw), then blocked for 1 hour with 300 μl/well of blocking buffer (PBS-tw supplemented with 5% Non-fat dried milk). Dilutions of undigested and digested proteins were prepared in a low-binding 96-well plate by serially diluting with blocking buffer. A 100 μL of each protein dilution was added to ELISA plate wells and incubated for 1 hour at room temperature on a plate shaker. The plates were washed and assayed for binding. An HRP-conjugated anti-camelid VHH antibody cocktail (GenScript Catalog #A02016) was diluted 10,000-fold in blocking buffer, and 100 μl/well was added to the ELISA plates. Plates were incubated for 30 minutes with shaking. ELISA plates were washed 2× with PBS-tw and 1× with PBS. HRP reaction was initiated by adding 100 μl/well of TMB Ultra ELISA substrate (Thermo Fisher) to each well. ELISA plates were developed by incubating for 5 minutes at room temperature and quenched with 50 μL of 1 M Hydrochloric acid. Absorbance was read at 450 nm with a Spectra Max M5 plate reader.

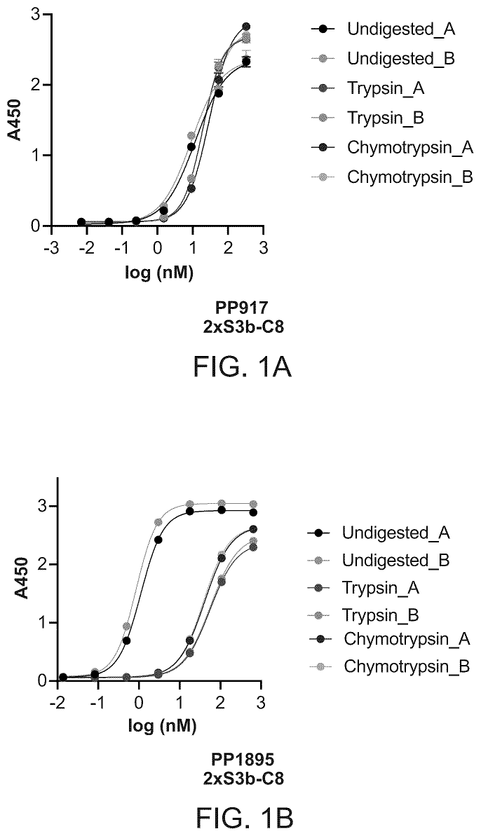

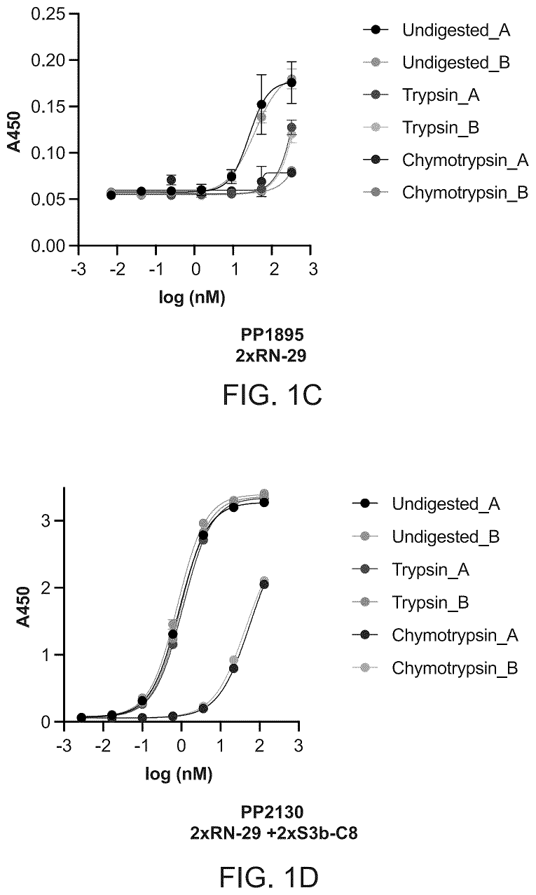

Results show that the pp 1895 construct is sensitive to both trypsin and chymotrypsin proteases: about 99% activity is lost after a 1 hr. incubation. Whereas the hydrabody complex, pp 2130, restores stability under trypsin condition, see FIG. 1A-FIG. 1D.

### Example 2—Mutations in smAKAP to Confer Trypsin and Chymotrypsin Resistance

Following the protease sensitivity study of Example 1 showing protease sensitivity in smAKAP, mutations in smAKAP were evaluated to determine if Trypsin and chymotrypsin resistance could be engineered into smAKAP.

To generate the mutant smAKAP clones, protein-protein interactions using structural and PISA analysis of the complex crystal structure (PDBID 5HVZ) were performed. Complex formation importance of Arginine (R) and Lysin (K) side chains, predicted to be Trypsin protease substrates, were assessed using structural tools. Similarly hydrophobic sidechains predicted to be chymotrypsin substrates were assessed and potential mutations were proposed to maintain complex formation. Various mutants were generated comprising mutations: C16S, C24S, E5D, Y6H, W22S, C24G, LAE, R9E, L4I, L10I, L19I and various combinations thereof as compared to the WT sequence of SEQ ID NO: 2.

TABLE 1
Exemplary mutant smAKAP sequences

| | | | SEQ ID |
|---|---|---|---|
| Description | Mutation | Sequence | NO |
| WT | N/A | TVILEYAHRLSQDILCDALQQW | 2 |
| smAKAP | | AC | |
| PP1895L | C16S_C24S | TVILEYAHRLSQDILSDALQQW | 5 |
| | | AS | |
| PP2452L | E5D_Y6H_C16S_W22S_C24G | TVILDHAHRLSQDILSDALQQS | 7 |
| | | AG | |
| PP2453L | L4E_E5D_Y6H_R9E_C16S_ | TVIEDHAHELSQDILSDALQQS | 15 |
| | W22S_C24G | AG | |
| PP2454L | L4E_C16S_W22S_C24G | TVIEEYAHRLSQDILSDALQQS | 17 |
| | | AG | |
| PP2455L | R9E_C16S_C24G | TVILEYAHELSQDILSDALQQW | 19 |
| | | AG | |
| PP2456L | R9E_C16S_W22S_C24G | TVILEYAHELSQDILSDALQQS | 21 |
| | | AG | |
| | L4I_L10I_C16S_L19I_C24G | TVIIEYAHRISQDILSDAIQQWA | 72 |
| | | G | |
| | L4I_R9E_L10I_C16S_L19I_ | TVIIEYAHEISQDILSDAIQQSAG | 73 |
| | W22S_C24G | | |
| PP6510L | L4I_R9E_C16S_C24G | TVIIEYAHELSQDILSDALQQW | 74 |
| | | AG | |

The exemplary mutant smAKAP linkers were utilized in scaffold constructs, see Table 2. see also FIG. 4.

TABLE 2
Exemplary mutant smAKAP clones, linkers, Cerberbody, and Hydrabody
Constructs

| | | Linker | | |
|---|---|---|---|---|
| Clone | Description | mutation | Linker Sequence | Sequence |
| NA | WT | NA | TVILEYAHRLSQDILCDALQQWAC (SEQ ID NO: | |
| | smAKAP | | 2) | |
| PP2451 | myctag-S3b- | NA | GGGGSGGGSG | MEQKLISEEDLSGGQVQLVE |
| | C8-5(4GS)- | | GGSGGGSGGG | SGGGLVQPGGSLRLSCAASG |
| | S3b-C8-H6 | | S | SIENIERMHWYRQAPGKQRE |
| | | | (SEQ ID NO: 3) | LVAVIDSGGTTRYADSVKGR |
| | | | | FTISRDNAKNTVYLQMNSLK |
| | | | | PEDTAVYYCAAVWLTNAES |
| | | | | TQEYWGQGTQVTVSSGGGG |
| | | | | SGGGSGGGSGGGSGGGSGS |
| | | | | QVQLVESGGGLVQPGGSLRL |
| | | | | SCAASGSIENIERMHWYRQA |
| | | | | PGKQRELVAVIDSGGTTRYA |
| | | | | DSVKGRFTISRDNAKNTVYL |
| | | | | QMNSLKPEDTAVYYCAAVW |
| | | | | LTNAESTQEYWGQGTQVTV |
| | | | | SSHHHHHH |
| | | | | (SEQ ID NO: 4) |
| PP1895 | S3B- | C70S_ | TVILEYAHRLS | MSGQVQLVESGGGLVQPGG |
| | C8_smAKAP- | C78S | QDILSDALQQW | SLRLSCAASGSIENIERMHWY |
| | C70S_C78S_ | | AS | RQAPGKQRELVAVIDSGGTT |
| | S3B- | | (SEQ ID NO: 5) | RYADSVKGRFTISRDNAKNT |
| | C8_H6 | | | VYLQMNSLKPEDTAVYYCA |
| | | | | AVWLTNAESTQEYWGQGTQ |
| | | | | VTVSSGGTVILEYAHRLSQD |
| | | | | ILSDALQQWASSGGGGSGG |
| | | | | GGGQVQLVESGGGLVQPGG |
| | | | | SLRLSCAASGSIENIERMHWY |
| | | | | RQAPGKQRELVAVIDSGGTT |
| | | | | RYADSVKGRFTISRDNAKNT |
| | | | | VYLQMNSLKPEDTAVYYCA |
| | | | | AVWLTNAESTQEYWGQGTQ |
| | | | | VTVSSGGSHHHHHH |
| | | | | (SEQ ID NO: 6) |
| PP2452 | myctag-S3b- | E59D_ | TVILDHAHRLS | MEQKLISEEDLSGGQVQLVE |
| | C8- | Y60H_ | QDILSDALQQS | SGGGLVQPGGSLRLSCAASG |
| | smAKAP_ | C70S_ | AG | SIENIERMHWYRQAPGKQRE |
| | E59D_Y60H_ | W76S_ | (SEQ ID NO: 7) | LVAVIDSGGTTRYADSVKGR |
| | C70S_W76S_ | C78G | | FTISRDNAKNTVYLQMNSLK |
| | C78S-S3b- | | | PEDTAVYYCAAVWLTNAES |
| | C8-H6 | | | TQEYWGQGTQVTVSSGGGG |
| | | | | STVILDHAHRLSQDILSDAL |
| | | | | QQSAGGGSGSQVQLVESGG |
| | | | | GLVQPGGSLRLSCAASGSIEN |
| | | | | IERMHWYRQAPGKQRELVA |
| | | | | VIDSGGTTRYADSVKGRFTIS |
| | | | | RDNAKNTVYLQMNSLKPED |
| | | | | TAVYYCAAVWLTNAESTQE |
| | | | | YWGQGTQVTVSSHHHHHH |
| | | | | (SEQ ID NO: 14) |
| PP2453 | myctag-S3b- | L58E_ | TVIEDHAHELS | MEQKLISEEDLSGGQVQLVE |
| | C8- | E59D_ | QDILSDALQQS | SGGGLVQPGGSLRLSCAASG |
| | smAKAP_ | Y60H_ | AG | SIENIERMHWYRQAPGKQRE |
| | L58E_E59D_ | R63E_ | (SEQ ID NO: 15) | LVAVIDSGGTTRYADSVKGR |
| | Y60H_R63E_ | C70S_ | | FTISRDNAKNTVYLQMNSLK |
| | C70S_W76S_ | W76S_ | | PEDTAVYYCAAVWLTNAES |
| | C78S- | C78G | | TQEYWGQGTQVTVSSGGGG |
| | S3b-C8-H6 | | | STVIEDHAHELSQDILSDAL |
| | | | | QQSAGGGSGSQVQLVESGG |
| | | | | GLVQPGGSLRLSCAASGSIEN |
| | | | | IERMHWYRQAPGKQRELVA |
| | | | | VIDSGGTTRYADSVKGRFTIS |
| | | | | RDNAKNTVYLQMNSLKPED |
| | | | | TAVYYCAAVWLTNAESTQE |
| | | | | YWGQGTQVTVSSHHHHHH |
| | | | | (SEQ ID NO: 16) |
| PP2454 | myctag-S3b- | L58E_ | TVIEEYAHRLS | MEQKLISEEDLSGGQVQLVE |
| | C8- | C70S_ | QDILSDALQQS | SGGGLVQPGGSLRLSCAASG |
| | smAKAP_ | W76S_ | AG | SIENIERMHWYRQAPGKQRE |
| | L58E_C70S_ | C78G | (SEQ ID NO: 17) | LVAVIDSGGTTRYADSVKGR |
| | W76S_C78S- | | | FTISRDNAKNTVYLQMNSLK |
| | S3b-C8-H6 | | | PEDTAVYYCAAVWLTNAES |
| | | | | TQEYWGQGTQVTVSSGGGG |
| | | | | STVIEEYAHRLSQDILSDAL |
| | | | | QQSAGGGSGSQVQLVESGG |
| | | | | GLVQPGGSLRLSCAASGSIEN |
| | | | | IERMHWYRQAPGKQRELVA |
| | | | | VIDSGGTTRYADSVKGRFTIS |
| | | | | RDNAKNTVYLQMNSLKPED |
| | | | | TAVYYCAAVWLTNAESTQE |
| | | | | YWGQGTQVTVSSHHHHHH |
| | | | | (SEQ ID NO: 18) |
| PP2455 | myctag-S3b- | R63E_ | TVILEYAHELS | MEQKLISEEDLSGGQVQLVE |
| | C8- | C70S_ | QDILSDALQQW | SGGGLVQPGGSLRLSCAASG |
| | smAKAP_ | C78G | AG | SIENIERMHWYRQAPGKQRE |
| | 6R3E_C70S_ | | (SEQ ID NO: 19) | LVAVIDSGGTTRYADSVKGR |
| | C78S-S3b- | | | FTISRDNAKNTVYLQMNSLK |
| | C8-H6 | | | PEDTAVYYCAAVWLTNAES |
| | | | | TQEYWGQGTQVTVSSGGGG |
| | | | | STVILEYAHELSQDILSDAL |
| | | | | QQWAGGGSGSQVQLVESGG |
| | | | | GLVQPGGSLRLSCAASGSIEN |
| | | | | IERMHWYRQAPGKQRELVA |
| | | | | VIDSGGTTRYADSVKGRFTIS |
| | | | | RDNAKNTVYLQMNSLKPED |
| | | | | TAVYYCAAVWLTNAESTQE |
| | | | | YWGQGTQVTVSSHHHHHH |
| | | | | (SEQ ID NO: 20) |
| PP2456 | myctag-S3b- | R63E_ | TVILEYAHELS | MEQKLISEEDLSGGQVQLVE |
| | C8- | C70S_ | QDILSDALQQS | SGGGLVQPGGSLRLSCAASG |
| | smAKAP_ | W76S_ | AG | SIENIERMHWYRQAPGKQRE |
| | 6R3E_C70S_ | C78G | (SEQ ID NO: 21) | LVAVIDSGGTTRYADSVKGR |
| | W76S_C78S- | | | FTISRDNAKNTVYLQMNSLK |
| | S3b-C8-H6 | | | PEDTAVYYCAAVWLTNAES |
| | | | | TQEYWGQGTQVTVSSGGGG |
| | | | | STVILEYAHELSQDILSDAL |
| | | | | QQSAGGGSGSQVQLVESGG |
| | | | | GLVQPGGSLRLSCAASGSIEN |
| | | | | IERMHWYRQAPGKQRELVA |
| | | | | VIDSGGTTRYADSVKGRFTIS |
| | | | | RDNAKNTVYLQMNSLKPED |
| | | | | TAVYYCAAVWLTNAESTQE |
| | | | | YWGQGTQVTVSSHHHHHH |
| | | | | (SEQ ID NO: 22) |
| PP917 | MBP- | | | MGKIEEGKLVIWINGDKGYN |
| | 5HVZ-S3b- | | | GLAEVGKKFEKDTGIKVTVE |
| | C8-H6 | | | HPDKLEEKFPQVAATGDGPD |
| | | | | IIFWAHDRFGGYAQSGLLAEI |
| | | | | TPDKAFQDKLYPFTWDAVR |
| | | | | YNGKLIAYPIAVEALSLIYNK |
| | | | | DLLPNPPKTWEEIPALDKELK |
| | | | | AKGKSALMFNLQEPYFTWPL |
| | | | | IAADGGYAFKYENGKYDIKD |
| | | | | VGVDNAGAKAGLTFLVDLIK |
| | | | | NKHMNADTDYSIAEAAFNK |
| | | | | GETAMTINGPWAWSNIDTSK |
| | | | | VNYGVTVLPTFKGQPSKPFV |
| | | | | GVLSAGINAASPNKELAKEF |
| | | | | LENYLLTDEGLEAVNKDKPL |
| | | | | GAVALKSYEEELVKDPRIAA |
| | | | | TMENAQKGEIMPNIPQMSAF |
| | | | | WYAVRTAVINAASGRQTVD |
| | | | | EALKDAQTGGGGSLRECELY |
| | | | | VQKHNIQALLKDSIVQLCTA |
| | | | | RPERPMAFLREYFEKLEKEE |
| | | | | AGGGGGQVQLVESGGGLVQ |
| | | | | PGGSLRLSCAASGSIENIERM |
| | | | | HWYRQAPGKQRELVAVIDS |
| | | | | GGTTRYADSVKGRFTISRDN |
| | | | | AKNTVYLQMNSLKPEDTAV |
| | | | | YYCAAVWLTNAESTQEYWG |
| | | | | QGTQVTVSSGGSHHHHHH |
| | | | | (SEQ ID NO: 23) |
| PP1113 | MBP- | | | MGKIEEGKLVIWINGDKGYN |
| | 5HVZ- | | | GLAEVGKKFEKDTGIKVTVE |
| | PAPA-RN- | | | HPDKLEEKFPQVAATGDGPD |
| | 29-H6 | | | IIFWAHDRFGGYAQSGLLAEI |
| | | | | TPDKAFQDKLYPFTWDAVR |
| | | | | YNGKLIAYPIAVEALSLIYNK |
| | | | | DLLPNPPKTWEEIPALDKELK |
| | | | | AKGKSALMFNLQEPYFTWPL |
| | | | | IAADGGYAFKYENGKYDIKD |
| | | | | VGVDNAGAKAGLTFLVDLIK |
| | | | | NKHMNADTDYSIAEAAFNK |
| | | | | GETAMTINGPWAWSNIDTSK |
| | | | | VNYGVTVLPTFKGQPSKPFV |
| | | | | GVLSAGINAASPNKELAKEF |
| | | | | LENYLLTDEGLEAVNKDKPL |
| | | | | GAVALKSYEEELVKDPRIAA |
| | | | | TMENAQKGEIMPNIPQMSAF |
| | | | | WYAVRTAVINAASGRQTVD |
| | | | | EALKDAQTGSLRECELYVQK |
| | | | | HNIQALLKDSIVQLCTARPER |
| | | | | PMAFLREYFEKLEKEEASPAP |
| | | | | APAGQVQLVESGGGLVQPG |
| | | | | GSLRLSCAASGYILSENLTA |
| | | | | WFRQAPGKAREVVATVTSG |
| | | | | DYTNYDDSVKGRFTISRDNA |
| | | | | KNTVYLQMNSLKPEDTAVY |
| | | | | YCAAGTSDWEVKWSHWGQ |
| | | | | GTQVTVSSGGSHHHHHH |
| | | | | (SEQ ID NO: 24) |
| PP6510 | MBP- | L58I_ | TVIIEYAHELSQ | MGKIEEGKLVIWINGDKGYN |
| | smAKAP_ | R63E_ | DILSDALQQWA | GLAEVGKKFEKDTGIKVTVE |
| | L58I_R63E_ | C70S_ | G | HPDKLEEKFPQVAATGDGPD |
| | C70S_C78G- | C78G | (SEQ ID NO: 74) | IIFWAHDRFGGYAQSGLLAEI |
| | PAPA- | | | TPDKAFQDKLYPFTWDAVR |
| | NANP- | | | YNGKLIAYPIAVEALSLIYNK |
| | StrepTag-6H | | | DLLPNPPKTWEEIPALDKELK |
| | | | | AKGKSALMFNLQEPYFTWPL |
| | | | | IAADGGYAFKYENGKYDIKD |
| | | | | VGVDNAGAKAGLTFLVDLIK |
| | | | | NKHMNADTDYSIAEAAFNK |
| | | | | GETAMTINGPWAWSNIDTSK |
| | | | | VNYGVTVLPTFKGQPSKPFV |
| | | | | GVLSAGINAASPNKELAKEF |
| | | | | LENYLLTDEGLEAVNKDKPL |
| | | | | GAVALKSYEEELVKDPRIAA |
| | | | | TMENAQKGEIMPNIPQMSAF |
| | | | | WYAVRTAVINAASGRQTVD |
| | | | | EALKDAQTTGSGSTVIIEYAH |
| | | | | ELSQDILSDALQQWAGSPAP |
| | | | | APAGNPNANPNANPNANPN |
| | | | | ANPNANPNANPNANPGGSW |
| | | | | SHPQFEKGGSHHHHHH |
| | | | | (SEQ ID NO: 76) |

In the pp 1895 construct the C78S can be interchanged with C78G as the position tolerates any small amino acid.

### Example 3—Mutant smAKAP Clones are Expressed in an E. Coli System

smAKAP mutants were generated according to the mutations described in Table 1 and evaluated if they could be expressed in an E. Coli System.

In brief, constructs were subcloned into a modified pET28 b (+) vector where the Kanamycin bacterial resistance gene was replaced with Ampicillin bacterial resistance gene. Sequence verified plasmids were transformed into BL21(DE3) Escherichia coli cells (New England Biolabs) and plated on LB medium supplemented with 100 μg mL-1 ampicillin. Proteins were expressed using autoinduction protocol where 100 mL of Super Broth media containing appropriate selection drug were inoculated with a single colony of individual transformants, shaken at 37° C. for 8 hours followed by 19° C. for 36 hours. Cultures were centrifuged and pellets stored at −20° C. until purification. Frozen pellets were thawed on ice and resuspended in 30 mL of 50 mM Tris pH 8.0, 300 mM NaCl, supplemented with pierce protease inhibitor tablets and 1 mM PMSF. Cells were lysed by high pressure homogenization (Microfluidics LM20) at 12,000 psi. The lysates were centrifuged at 18500 g at 4° C. on tabletop Centrifuge (Eppendorf Centrifuge 5810R) for 30 minutes. Supernatant were used in further processing. Recombinant proteins were purified from the clarified lysate by affinity chromatography using 5 mL HisTrap columns (Cytiva, cat. no. 17524802) equilibrated with 50 mM Tris pH 8.0, 300 mM NaCl and 20 mM imidazole (Thermo Fisher Scientific, cat. no. O3196-500). Hexa-Histidine tagged and HisTrap-purified samples were further purified using AKTA Pure FPLC-based size-exclusion chromatography Superdex 200 increase 10/300 (Cytiva, cat. no. 28990944). Protein purity and sizing were assayed by size-exclusion chromatography on analytical Superdex 200 increase 10/300 (Cytiva, cat. no. 28990944) where 100 μL of purified protein at ˜ 1 mg/mL were run in 1×PBS buffer at room temperature. Purified protein samples were also assessed on SDS-PAGE under reducing and non-reducing conditions. 3 μg protein samples were incubated at 70° C. in LDS loading buffer with or without 50 mM dithiothreitol (DTT) reducing agent (Thermo Fisher Scientific, cat. no. R0862).

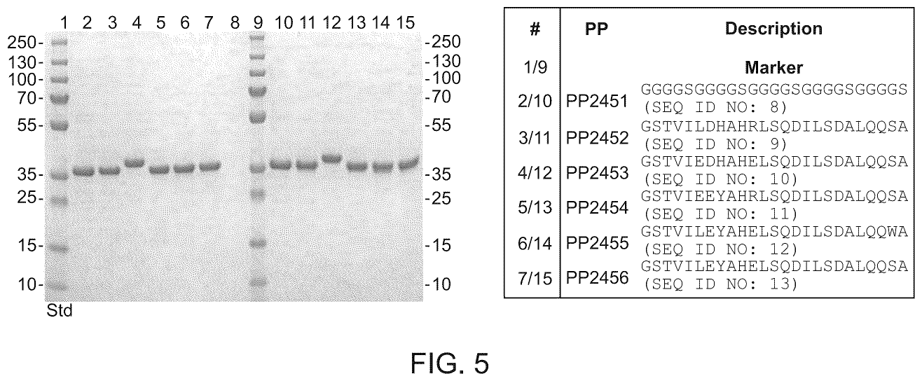

Results show that all of the mutant smAKAP clones expressed in the E. Coli, see FIG. 5.

### Example 4—Evaluation of smAKAP Mutants and Protease Resistance

### Trypsin Resistance

smAKAP mutants were generated, according to the method of Example 1, with an R63E substitution to evaluate if the mutation confers Trypsin resistance in the mutant smAKAP peptides

Clones pp 2451, pp 1895, and pp 2455 were evaluated for Trypsin resistance utilizing similar methods as described in Example 1.

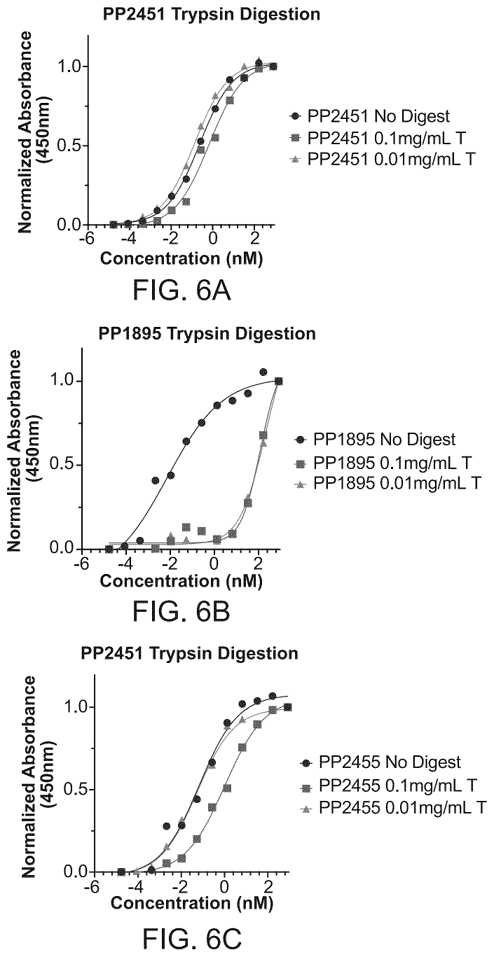

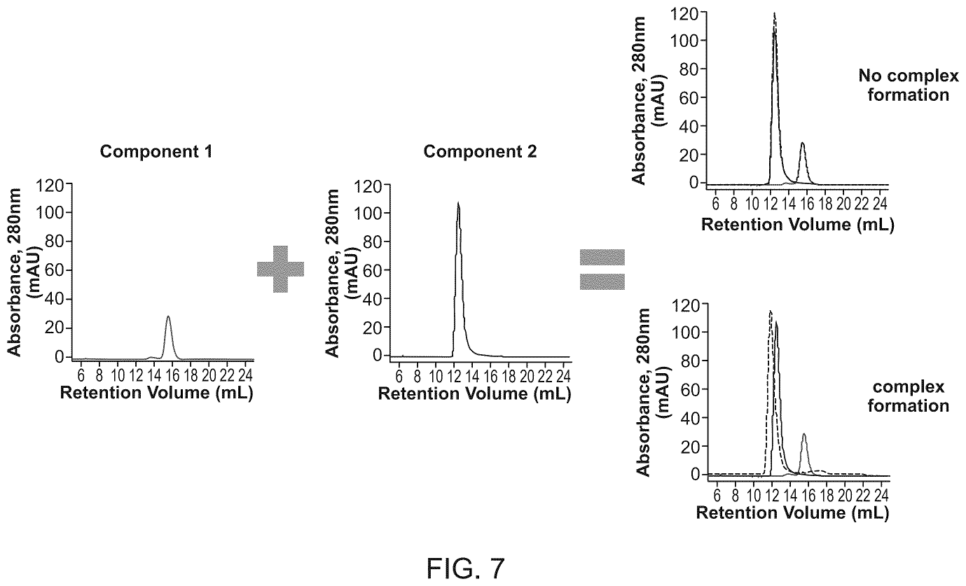

Results indicate that the R63E mutation rescues WT smAKAP sensitivity to trypsin, see FIG. 6A-FIG. 6C.

### Chymotrypsin Resistance

smAKAP mutants generated according to Example 1 were also evaluated for their ability to resistance chymotrypsin cleavage. Specifically, clones pp 2451, pp 1895, pp 2455, pp 2456, pp 2454, pp 2452, and pp 2453 were subjected to the assay utilizing similar methods as described in Example 1.

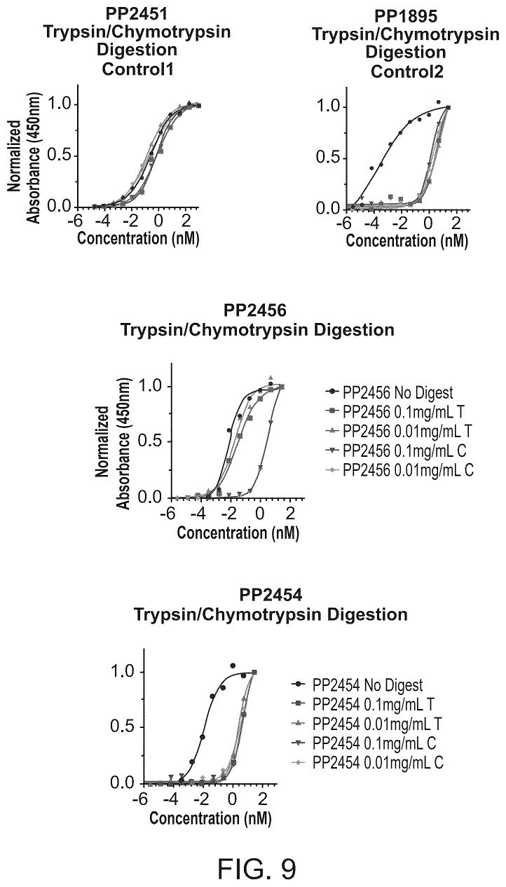

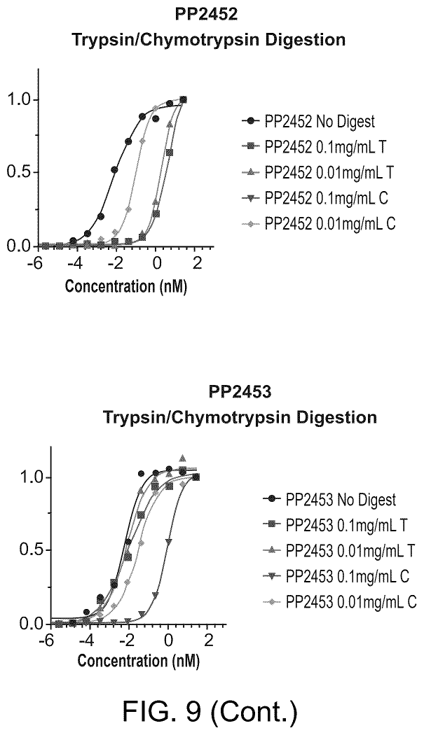

Results indicate that combining hydrophobic mutations, such as in pp 2453, yields the highest improved resistance, see FIG. 9. Furthermore, in cases of detected chymotrypsin sensitivity it was mainly due to the presence of the smAKAP linker. However, alternate linkers can be utilized with the compositions of the disclosure.

### Example 5—Complex Formation Analysis

### Generation of Dimeric Complexes

Dimers of smAKAP and 5HVZ were generated and evaluated for their ability to form scaffold complexes.

Dimer formation through the 5HVZ motif is due to protein-protein interactions between three α-helical chains, placing the monomeric polypeptides in antiparallel conformation. In addition, asymmetric disulfide bond formation between the protomers further strengthens the dimer structure. Dimeric proteins are expressed from clones that genetically encode a single polypeptide containing a 5HVZ dimerization motif fused to the protein of interest. The dimerization motif (5HVZ) can be fused on both N and C-termini to generate homo- or heterodimeric protein constructs. Engineering VHH constructs using the 5HVZ dimerization motif for Spirulina expression was shown to increase binding avidity to antigens of interest. Dimer formation by 5HVZ motif results in a protein fold that serves as a docking platform facilitating interaction between PKA and the membrane-attached A-kinase anchoring protein (AKAP). A small alpha-helical peptide in the AKAP, smAKAP, docks on the 5HVZ platform.

### Complex Formation Evaluation

To verify if smAKAP mutants retained the ability to form a scaffold complex, a complex formation analysis was completed utilizing the pp 2451, pp 1895, pp 2456 (W76S), and pp 2455 (R63E mutant) clones.

To assess higher-order complex formation (Hydrabody), purified dimeric proteins (PP917 and pp 1113), where homodimerization is achieved through the 5HVZ scaffold, were run on analytical Superdex 200 increase 10/300 (Cytiva, cat. no. 28990944). The proteins were run alone or in a 1:2 molar ratio with the proteins from smAKAP mutant and control clones. Complex formation efficiencies were compared by comparing retention volume and protein absorbance from the dimeric and smAKAP-containing constructs assessed alone. smAKAP-containing constructs that form a complex with 5HVZ dimeric scaffold-containing constructs exhibit a shift to smaller retention volume and increased absorbance for the complex. In addition, complex forming constructs show a significant reduction in absorbance peak height at the retention volume where smAKAP-containing constructs were expected to elute.

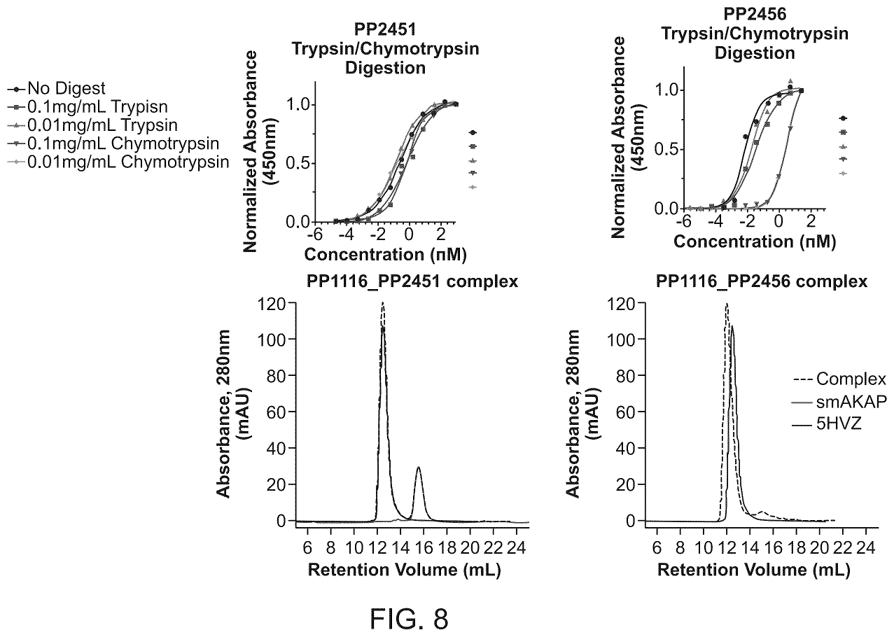

Results show that R63E and W76S substitutions did not affect complex formation in scaffolds comprising the mutant smAKAP, see FIG. 8. Therefore, not only was complex formation not affected, the mutated smAKAP exhibited improved protease resistance as compared to control.

Utilizing similar methods as described above, an additional study was completed utilizing alternate mutant clones: pp 2451, pp 1895, pp 2456, pp 2454, pp 2452, and pp 2453.

Specifically, the crystal structure for the smAKAP and dimer interface was calculated based on the crystal structure (PDB ID 5HVZ) using the web based interactive analysis tool PDBePISA (PISA). The interface formed between the dimerization motif “5HVZ” and the smAKAP docking peptide was assessed and scored for stabilizing physicochemical properties. The PISA analysis tool uses physicochemical properties, including solvation energy, interface area, hydrogen bonds, free energy of formation, and salt bridge formation to predict strength of complex formation. The summary data presented in Tables 3-4 shows the hydrogen bond formation or salt bridge formation by the amino acid chain indicated (HSDC column), solvent accessible surface area (ASA), buried surface area (BSA), Buried area percentage (one bar per 10%) and solvation energy effect (AiG). The analysis was used to determine the effect of mutations of complex formation.

TABLE 3
Interaction between smAKAP peptide and 5HVZ dimer protomer A

| ## | Structure 1 | HSDC | ASA | BSA | ΔiG | |
|---|---|---|---|---|---|---|
| 1 | C:THR 55 | | 132.99 | 0.00 | 0.00 | Solvent-accessible |
| | | | | | | residues |
| 2 | C:VAL 56 | | 114.52 | 0.00 | 0.00 | Solvent-accessible |
| | | | | | | residues |
| 3 | C:ILE 57 | | 133.51 | 48.55 \|\|\|\| | 0.78 | |
| 4 | C:LEU 58 | | 135.71 | 41.33 \|\|\|\| | 0.65 | |
| 5 | C:GLU 59 | | 102.08 | 0.00 | 0.00 | Solvent-accessible |
| | | | | | | residues |
| 6 | C:TYR 60 | | 138.80 | 21.42 \|\| | 0.34 | |
| 7 | C:ALA 61 | | 48.61 | 48.61 \|\|\|\|\|\|\|\|\|\| | 0.70 | |
| 8 | C:HIS 62 | | 109.48 | 26.49 \|\|\| | 0.15 | |
| 9 | C:ARG 63 | | 164.72 | 0.00 | 0.00 | Solvent-accessible |
| | | | | | | residues |
| 10 | C:LEU 64 | | 69.65 | 18.91 \|\|\| | 0.30 | |
| 11 | C:SER 65 | | 56.44 | 56.44 \|\|\|\|\|\|\|\|\|\| | 0.56 | |
| 12 | C:GLN 66 | H | 94.75 | 20.97 \|\|\| | 0.17 | |
| 13 | C:ASP 67 | | 74.90 | 0.00 | 0.00 | |
| 14 | C:ILE 68 | | 102.18 | 27.06 \|\|\| | 0.43 | |
| 15 | C:LEU 69 | | 111.17 | 90.08 \|\|\|\|\|\|\|\|\| | 1.44 | |
| 16 | C:CYS 70 | | 85.33 | 0.00 | 0.00 | Solvent-accessible |
| | (Mutated to | | | | | residues |
| | Ser | | | | | |
| 17 | C:ASP 71 | | 92.34 | 0.00 | 0.00 | Solvent-accessible |
| | | | | | | residues |
| 18 | C:ALA 72 | | 50.27 | 0.00 | 0.00 | Solvent-accessible |
| | | | | | | residues |
| 19 | C:LEU 73 | | 116.42 | 75.90 \|\|\|\|\|\|\| | 1.00 | |
| 20 | C:GLN 74 | | 81.25 | 0.00 | 0.00 | Solvent-accessible |
| | | | | | | residues |
| 21 | C:GLN 75 | | 147.99 | 0.00 | 0.00 | Solvent-accessible |
| | | | | | | residues |
| 22 | C:TRP 76 | | 90.60 | 23.82 \|\|\| | 0.06 | |
| 23 | C:ALA 77 | | 126.60 | 24.27 \|\| | 0.39 | |

HSDC
H = Hydrogen bond
S = Salt bridge
ASA = Accessible Surface Area, Å²
BSA = Buried Surface Area, Å²
ΔiG = Solvation energy effect, kcal/mol
|||| = Buried area percentage, one bar per 10%

TABLE 4
Interaction between smAKAP peptide and 5HVZ dimer protomer B

| ## | Structure 1 | HSDC | ASA | BSA | ΔiG | |
|---|---|---|---|---|---|---|
| 1 | C:THR 55 | | 132.99 | 0.00 | 0.00 | Solvent-accessible |
| | | | | | | residues |
| 2 | C:VAL 56 | | 114.52 | 35.83 \|\|\|\| | 0.57 | |
| 3 | C:ILE 57 | | 133.51 | 59.14 \|\|\|\|\| | 0.95 | |
| 4 | C:LEU 58 | | 135.71 | 0.00 | 0.00 | Solvent-accessible |
| | | | | | | residues |
| 5 | C:GLU 59 | | 102.08 | 0.00 | 0.00 | Solvent-accessible |
| | | | | | | residues |
| 6 | C:TYR 60 | | 138.80 | 87.14 \|\|\|\|\|\|\| | 0.68 | |
| 7 | C:ALA 61 | | 48.61 | 0.00 | 0.00 | Solvent-accessible |
| | | | | | | residues |
| 8 | C:HIS 62 | | 109.48 | 0.00 | 0.00 | Solvent-accessible |
| | | | | | | residues |
| 9 | C:ARG 63 | | 164.72 | 0.00 | 0.00 | Solvent-accessible |
| | | | | | | residues |
| 10 | C:LEU 64 | | 69.65 | 46.69 \|\|\|\|\|\|\| | 0.75 | |
| 11 | C:SER 65 | | 56.44 | 0.00 | 0.00 | Solvent-accessible |
| | | | | | | residues |
| 12 | C:GLN 66 | | 94.75 | 0.00 | 0.00 | Solvent-accessible |
| | | | | | | residues |
| 13 | C:ASP 67 | | 74.90 | 9.35 \|\| | 0.15 | |
| 14 | C:ILE 68 | | 102.18 | 75.12 \|\|\|\|\|\|\|\| | 1.20 | |
| 15 | C:LEU 69 | | 111.17 | 21.09 \|\| | 0.34 | |
| 16 | C:CYS 70 (Mutated | | 85.33 | 0.00 | 0.00 | Solvent-accessible |
| | to Ser | | | | | residues |
| 17 | C:ASP 71 | HS | 92.34 | 34.91 \|\|\|\| | 0.02 | |
| 18 | C:ALA 72 | | 50.27 | 48.84 \|\|\|\|\|\|\|\|\|\| | 0.70 | |
| 19 | C:LEU 73 | | 116.42 | 20.43 \|\| | 0.33 | |
| 20 | C:GLN 74 | | 81.25 | 0.00 | 0.00 | Solvent-accessible |
| | | | | | | residues |
| 21 | C:GLN 75 | H | 147.99 | 34.68 \|\|\| | 0.24 | |
| 22 | C:TRP 76 | | 90.60 | 35.46 \|\|\|\| | 0.56 | |
| 23 | C:ALA 77 | | 126.60 | 0.00 | 0.00 | Solvent-accessible |
| | | | | | | residues |

HSDC
H = Hydrogen bond
S = Salt bridge
ASA = Accessible Surface Area, Å²
BSA = Buried Surface Area, Å²
ΔiG = Solvation energy effect, kcal/mol
|||| = Buried area percentage, one bar per 10%

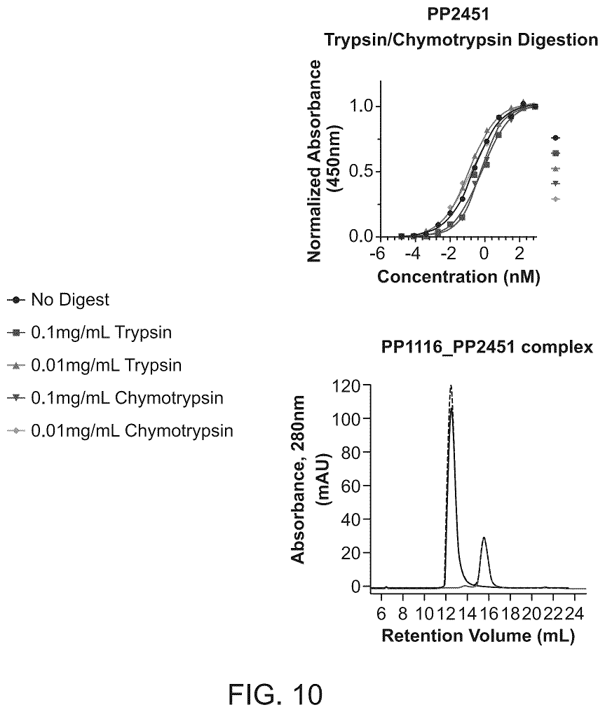

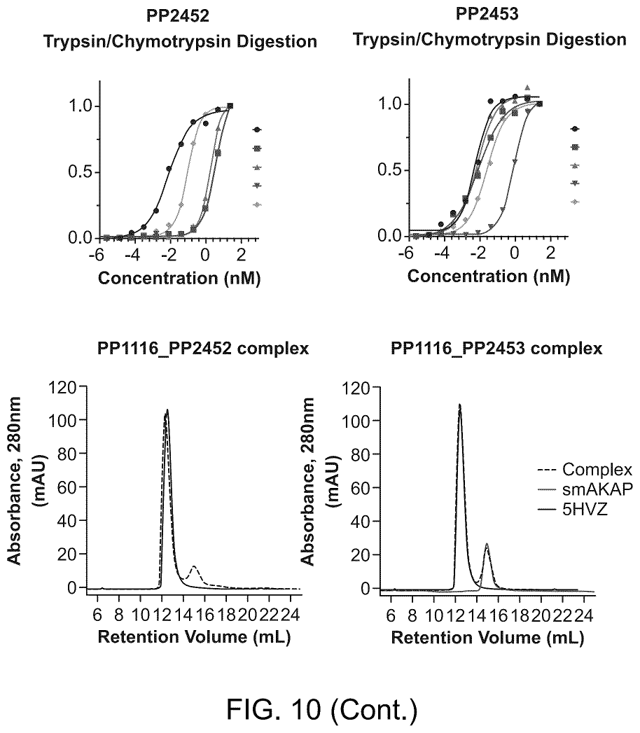

These results indicate that the Y60H mutation shows the largest effect on complex formation, see also FIG. 10.

### Example 6—Increasing Complex Stability by Way of smAKAP Mutations

Additional mutations were evaluated for their ability to stabilize smAKAP complexes. Clones PP2451-PP2456 were subjected to a stability assay. To assess stability of purified proteins, protein samples were assessed by SDS-PAGE gel under non-reducing or reducing (50 mM DTT) conditions). The same lots of proteins were also stored at 4° C. for up to 6 weeks and assessed for stability by SDS-PAGE gel under non-reducing or reducing (50 mM DTT) conditions). A comparative analysis of the SDS-PAGE gel indicated decreased fragmentation for some of the smAKAP mutant linker clones.

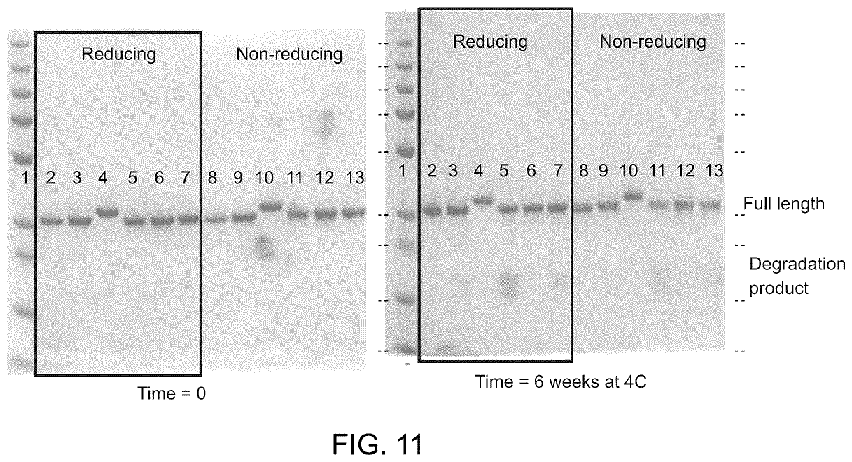

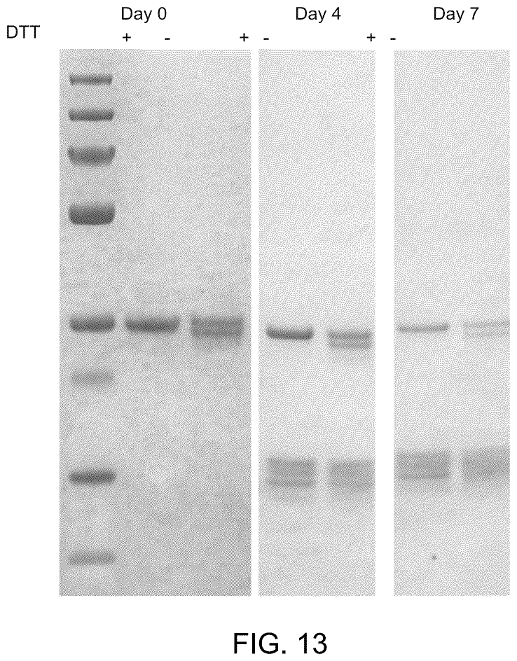

Results show that the clones pp 2453 and pp 2455, which contain R63E mutation, are more stable, see FIG. 11 vs. FIG. 13.

### Example 7—smAKAP Linker Containing Constructs Express in Spirulina

smAKAP mutants were generated according to the mutations described in Table 1 and evaluated if they could be expressed in an Spirulina System.

In brief, constructs were subcloned into a modified pET28 b (+) vector where the Kanamycin bacterial resistance gene was replaced with Ampicillin bacterial resistance gene. Sequence verified plasmids were transformed into Spirulina. Proteins were expressed, isolated, and assayed by size-exclusion chromatography on analytical Superdex 200 increase 10/300 (Cytiva, cat. no. 28990944) where 100 μL of purified protein at about 1 mg/mL were run in 1×PBS buffer at room temperature. Purified protein samples were also assessed on SDS-PAGE under reducing and non-reducing conditions. 3 μg protein samples were incubated at 70° C. in LDS loading buffer with or without 50 mM dithiothreitol (DTT) reducing agent (Thermo Fisher Scientific, cat. no. R0862).

TABLE 5
smAKAP linker Containing Constructs Express in Spirulina

| | | Total | Sol. | Sol. |
|---|---|---|---|---|
| | | Target, % | Target, % | Target, % |
| Sample ID | Description | DW | DW | TSP |
| SP2245-001 | S3B-C8_smAKAP- | 1.88 | >2 | >2.91 |
| | C16S_S3B-C8_H6 | | | |
| SP2246-001 | S3B-C8_smAKAP- | 0.92 | 1.06 | 1.5 |
| | C16S_R2B-D5_H6 | | | |

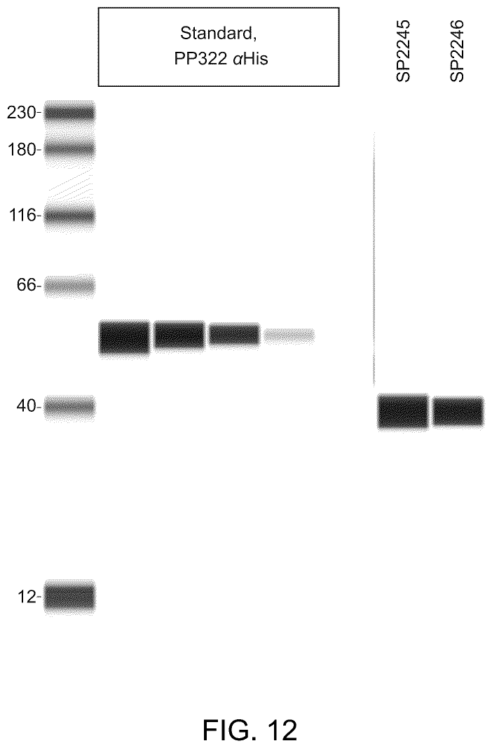

Results show that smAKAP linker Containing Constructs Express in Spirulina, see FIG. 12.

### Example 8—Higher Order Complex Formation Via an smAKAP Mutant

Higher order complex formation using the 5HVZ dimerization domain can be achieved by complexing with proteins linked by an smAKAP peptide. The effects of amino acid substitutions of the smAKAP peptide on complex formation were evaluated via studies comparing WT smAKAP peptide (SEQ ID NO: 2) vs. the pp 6510 mutant (SEQ ID NO: 74).

In brief, proteins linked through the pp 6510 mutant were cloned into the pET28 expression vector (SEQ ID NO: 75) and expressed in E. coli cells. Purified proteins were assessed by size exclusion chromatography (SEC). To analyze complex formation, purified dimeric proteins (PP6510 and PP1116), where homodimerization is achieved through the 5HVZ scaffold, were run on analytical Superdex 200 increase 10/300 (Cytiva, cat. no. 28990944). The proteins were run alone or in a 1:1.2 molar ratio with the proteins from smAKAP mutant and control clones. Complex formation efficiencies were compared by comparing retention volume and protein absorbance from the dimeric and smAKAP-containing constructs assessed alone.

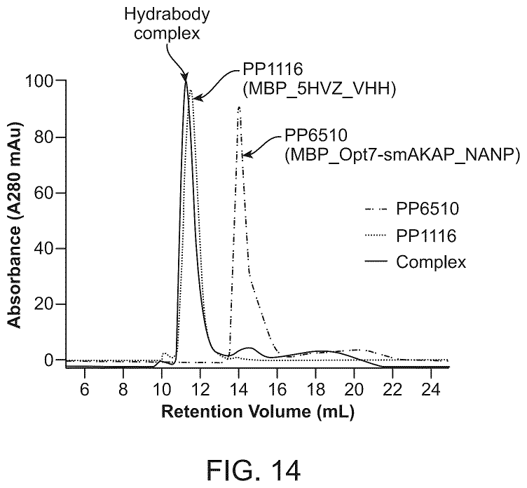

Results show Applicant's success in utilizing the pp 6510 mutant to form high order oligomers with 5HVZ-mediated homodimer proteins, see FIG. 14.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

Provided compositions may comprise the amino acid sequences as described in International Patent Application Nos. PCT/US2020/040794 and PCT/US2021/065138.

1. A recombinant small-membrane A-kinase anchoring protein (smAKAP) peptide, said smAKAP peptide comprising at least one amino acid substitution selected from the group consisting of C16S, C24S, E5D, Y6H, W22S, C24G, L4E, R9E, L4I, L10I, and L19I, wherein the substitutions are based on alignment with SEQ ID NO: 2.

2. The recombinant smAKAP peptide of claim 1, comprising amino acid substitution L4E based on alignment with SEQ ID NO: 2.

3. The recombinant smAKAP peptide of claim 1, comprising amino acid substitution R9E based on alignment with SEQ ID NO: 2.

4. The recombinant smAKAP peptide of claim 1, comprising amino acid substitutions L4E and R9E based on alignment with SEQ ID NO: 2.

5. The recombinant smAKAP peptide of claim 1, wherein the recombinant smAKAP peptide exhibits resistance to protease cleavage compared to SEQ ID NO: 2, wherein resistance to protease cleavage is determined by reduced detection of cleavage products when exposed to a solvent comprising a protease for one hour.

6. The recombinant smAKAP peptide of claim 5, wherein at most about 5%, 10%, 20%, 30%, 40%, 50%, or 60% of the smAKAP peptide is cleaved after one hour.

7. The recombinant smAKAP peptide of claim 1, wherein the recombinant smAKAP peptide is linked to a heterologous moiety.

8. The recombinant smAKAP peptide of claim 7, wherein the recombinant smAKAP peptide is linked to a second heterologous moiety.

9. The recombinant smAKAP peptide of claim 8, wherein the heterologous moiety and the second heterologous moiety are the same.

10. The recombinant smAKAP peptide of claim 8, wherein the heterologous moiety and the second heterologous moiety are different.

11. The recombinant smAKAP peptide of claim 7, wherein the heterologous moiety is a binding agent is selected from the group consisting of: fab′, F(ab′) 2, fv, domain antibody (dAb), complementarity Determining Region (CDR) fragment, CDR-grafted antibody, single chain antibodies (scFv), single chain antibody fragment, chimeric antibody, diabody, triabody, tetrabody, minibody, linear antibody, intrabody, nanobody (single domain antibody), small Modular Immunopharmaceuticals (SMIPs), antigen-binding domain immunoglobulin fusion protein, and VHH.

12. The recombinant smAKAP peptide of claim 1, wherein the recombinant smAKAP peptide is linked to a VHH antibody.

13. The recombinant smAKAP peptide of claim 1, wherein the recombinant smAKAP peptide is linked to two VHH antibodies, wherein a first VHH is at a C-terminus and a second VHH is at an N-terminus of the recombinant smAKAP peptide.

14. The recombinant smAKAP peptide of claim 12, wherein the VHH antibody comprises a sequence selected from the group consisting of SEQ ID NO: 25-67.

15. The recombinant smAKAP peptide of claim 1, said recombinant smAKAP in contact with a scaffold having at least 85%, 90%, 95%, 96%, 97%, 98%, 99%, or 100% identity to a sequence of SEQ ID NO: 4, 6, 14, 16, 18, 20, 22, 23, 24, and 76.

16. A recombinant Spirulina comprising the recombinant smAKAP peptide of claim 1.

17. The recombinant Spirulina of claim 16, wherein the Spirulina is from a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. jusiformis, A. ghannae, A. gigantean, A. gomontiana, A. gomontiana var. crassa, A. indica, A. jenneri var. platensis, A. jenneri Stizenberger, A. jenneri f purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoidesf. tenuis, A. spirulinoides, A. subsalsa, A. subtilissima, A. tenuis, A. tenuissima, and A. versicolor.

18. A vector encoding the recombinant smAKAP peptide of claim 1.

19. A method of treatment comprising administering the recombinant peptide of claim 1 to a subject in need thereof, wherein the subject comprises a bacterial or viral infection.

20. The method of claim 19, wherein the infection is caused by a pathogen selected from the group consisting of: E. coli, Enterotoxigenic E. coli (ETEC), anthrax, EHEC, EAEC, Shigella, Mycobacterium, Streptococcus, Staphylococcus, Shigella, Campylobacter, Salmonella, Clostridium, Corynebacterium, Pseudomonas, Neisseria, Listeria, Vibrio, Bordetelia, Legionella, bacteriophage, RNA bacteriophage (e.g. MS2, AP205, PP7 and QP), Helicobacter pylori, Infectious Hematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, White Spot Syndrome Virus, a coronavirus, SARS, MERS, SARS-COV-2, Aspergillus, Candida, Blastomyces, Coccidioides, Cryptococcus, Histoplasma, Plasmodium, P. falciparum, P. malariae, P. ovale, P. vivax, Trypanosoma, Toxoplasma, Giardia, Leishmania Cryptosporidium, helminthic parasites: Trichuris spp., Enterobius spp., Ascaris spp., Ancylostoma spp. and Necatro spp., Strongyloides spp., Dracunculus spp., Onchocerca spp. Wuchereria spp., Taenia spp., Echinococcus spp., Diphyllobothrium spp., Fasciola spp., and Schistosoma spp.


SEQUENCE LISTING (76 sequences)

SEQ ID NO: 1 - protein, 25 aa, synthetic construct
GSTVILEYAHRLSQDILSDALQQWA

SEQ ID NO: 2 - protein, 24 aa, Homo sapiens
TVILEYAHRLSQDILCDALQQWAC

SEQ ID NO: 3 - protein, 21 aa, synthetic construct
GGGGSGGGSGGGSGGGSGGGS

SEQ ID NO: 4 - protein, 283 aa, synthetic construct
MEQKLISEEDLSGGQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSGGGGSGGGSGGGSGGGSGGGSGSQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSHHHHHH

SEQ ID NO: 5 - protein, 24 aa, synthetic construct
TVILEYAHRLSQDILSDALQQWAS

SEQ ID NO: 6 - protein, 289 aa, synthetic construct
MSGQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSGGTVILEYAHRLSQDILSDALQQWASSGGGGSGGGGGQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSGGSHHHHHH

SEQ ID NO: 7 - protein, 24 aa, synthetic construct
TVILDHAHRLSQDILSDALQQSAG

SEQ ID NO: 8 - protein, 25 aa, synthetic construct
GGGGSGGGGSGGGGSGGGGSGGGGS

SEQ ID NO: 9 - protein, 25 aa, synthetic construct
GSTVILDHAHRLSQDILSDALQQSA

SEQ ID NO: 10 - protein, 25 aa, synthetic construct
GSTVIEDHAHELSQDILSDALQQSA

SEQ ID NO: 11 - protein, 25 aa, synthetic construct
GSTVIEEYAHRLSQDILSDALQQSA

SEQ ID NO: 12 - protein, 25 aa, synthetic construct
GSTVILEYAHELSQDILSDALQQWA

SEQ ID NO: 13 - protein, 25 aa, synthetic construct
GSTVILEYAHELSQDILSDALQQSA

SEQ ID NO: 14 - protein, 294 aa, synthetic construct
MEQKLISEEDLSGGQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSGGGGSTVILDHAHRLSQDILSDALQQSAGGGSGSQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSHHHHHH

SEQ ID NO: 15 - protein, 24 aa, synthetic construct
TVIEDHAHELSQDILSDALQQSAG

SEQ ID NO: 16 - protein, 294 aa, synthetic construct
MEQKLISEEDLSGGQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSGGGGSTVIEDHAHELSQDILSDALQQSAGGGSGSQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSHHHHHH

SEQ ID NO: 17 - protein, 24 aa, synthetic construct
TVIEEYAHRLSQDILSDALQQSAG

SEQ ID NO: 18 - protein, 294 aa, synthetic construct
MEQKLISEEDLSGGQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSGGGGSTVIEEYAHRLSQDILSDALQQSAGGGSGSQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSHHHHHH

SEQ ID NO: 19 - protein, 24 aa, synthetic construct
TVILEYAHELSQDILSDALQQWAG

SEQ ID NO: 20 - protein, 294 aa, synthetic construct
MEQKLISEEDLSGGQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSGGGGSTVILEYAHELSQDILSDALQQWAGGGSGSQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSHHHHHH

SEQ ID NO: 21 - protein, 24 aa, synthetic construct
TVILEYAHELSQDILSDALQQSAG

SEQ ID NO: 22 - protein, 294 aa, synthetic construct
MEQKLISEEDLSGGQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSGGGGSTVILEYAHELSQDILSDALQQSAGGGSGSQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSHHHHHH

SEQ ID NO: 23 - protein, 555 aa, synthetic construct
MGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGGGGSLRECELYVQKHNIQALLKDSIVQLCTARPERPMAFLREYFEKLEKEEAGGGGGQVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSSGGSHHHHHH

SEQ ID NO: 24 - protein, 554 aa, synthetic construct
MGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGSLRECELYVQKHNIQALLKDSIVQLCTARPERPMAFLREYFEKLEKEEASPAPAPAGQVQLVESGGGLVQPGGSLRLSCAASGYILSENLTAWFRQAPGKAREVVATVTSGDYTNYDDSVKGRFTISRDNAKNTVYLQMNSLKPEDTAVYYCAAGTSDWEVKWSHWGQGTQVTVSSGGSHHHHHH

SEQ ID NO: 25 - protein, 135 aa, synthetic construct
QVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSEPKTPKPQ

SEQ ID NO: 26 - protein, 119 aa, synthetic construct
QVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSEPKTPKPQ

SEQ ID NO: 27 - protein, 123 aa, synthetic construct
VQLVESGGGLVQAGGSLRLSCAASGLMFGAMTMGWYRQAPGKEREMVAYITAGGTESYSESVKGRFTISRINANNMVYLQMTNLKVEDTAVYYCNAHNFWRTSRNWGQGTQVTVSSEPKTPKP

SEQ ID NO: 28 - protein, 131 aa, synthetic construct
VQLVESGGGLVQAGDSLTLSCAASESTFNTFSMAWFRQAPGKEREYVAAFSRSGGTTNYADSVKGRATISTDNAKNTVYLHMNSLKPEDTAVYFCAADRPAGRAYFQSRSYNYWGQGTQVTVSSAHHSEDP

SEQ ID NO: 29 - protein, 125 aa, synthetic construct
VQLVESGGGSVQIGGSLRLSCVASGFTFSKNIMSWARQAPGKGLEWVSTISIGGAATSYADSVKGRFTISRDNANDTLYLQMNNLKPEDTAVYYCSRGPRTYINTASRGQGTQVTVSSEPKTPKP

SEQ ID NO: 30 - protein, 129 aa, synthetic construct
VQLVESGGGLVQAGGSLRLSCAAPGLTFTSYRMGWFRQAPGKEREYVAAITGAGATNYADSAKGRFTISKNNTASTVHLQMNSLKPEDTAVYYCAASNRAGGYWRASQYDYWGQGTQVTVSSAHHSEDP

SEQ ID NO: 31 - protein, 135 aa, synthetic construct
QVQLVESGGGLVQPGGSLRLSCAASGFSLDYYGIGWFRQAPGKERQEVSYISASAKTKLYSDSVKGRFTISRDNAKNAVYLEMNSLKREDTAVYYCARRRFDASASNRWLAADYDYWGQGTQVTVSSEPKTPKPQ

SEQ ID NO: 32 - protein, 123 aa, synthetic construct
QVQLVESGGGLVQAGGSLRLSCVSSERNPGINAMGWYRQAPGSQRELVAIWQTGGSLNYADSVKGRFTISRDNLKNTVYLQMNSLKPEDTAVYYCYLKKWRDQYWGQGTQVTVSSEPKTPKPQ

SEQ ID NO: 33 - protein, 124 aa, synthetic construct
QVQLVESGGGLVEAGGSLRLSCVVTGSSFSTSTMAWYRQPPGKQREWVASFTSGGAIKYTDSVKGRFTMSRDNAKKMTYLQMENLKPEDTAVYYCALHNAVSGSSWGRGTQVTVSSEPKTPKPQ

SEQ ID NO: 34 - protein, 131 aa, synthetic construct; identical to SEQ ID NO: 28

SEQ ID NO: 35 - protein, 121 aa, synthetic construct
VQLVESGGGLVQAGGSLRLSCVGSGRNPGINAMGWYRQAPGSQRELVAVWQTGGSTNYADSVKGRFTISRDNLKNTVYLQMNSLKPEDTAVYYCYLKKWRDEYWGQGTQVTVSSAHHSEDP

SEQ ID NO: 36 - protein, 124 aa, synthetic construct
VQLVESGGGLVQAGESLRLSCVVSESIFRINTMGWYRQTPGKQREVVARITLRNSTTYADSVKGRFTISRDDAKNTLYLKMDSLKPEDTAVYYCHRYPLIFRNSPYWGQGTQVTVSSEPKTPKP

SEQ ID NO: 37 - protein, 122 aa, synthetic construct
VQLVESGGGLVQAGESLRLSCVVSESIFRINTMGWYRQTPGKQREVVARITLRNSTTYADSVKGRFTISRDDAKNTLYLKMDSLKPEDTAVYYCHRYPLIFRNSPYWGQGTQVTVSSEPKTP

SEQ ID NO: 38 - protein, 131 aa, synthetic construct
QVQLQESGGGLVQPGGSLRLSCAASESTPSINTMGWYRQAPGKERELVATITSGGMTNYADSVKGRFTISRDNGKNTVYLQMNSLEPGDTAVYYCNLKRRDLQARFGGYWGQGTQVTVSSEPKTQTTTSGR

SEQ ID NO: 39 - protein, 132 aa, synthetic construct
QVKLQQSGGGLVQPGGSLRLSCAASESTISINTLGWYRQAPGNQRELVATITTGGTTNYADSVKGRFTISRDNAKNTVYLQMNNLEPGDTAVYYCNLKRRDLQSRFGGYWGQGTQVTVSSEPQDTKTTTSGR

SEQ ID NO: 40 - protein, 131 aa, synthetic construct
QVQLQQSGGGLVQPGGSLRLSCVASESTVSINIMGWYRQAPGKQRELVATITTGGTTNYADSVKGRFTISRDNAKNTVYLQMNSLEPEDTAVYYCNLKRRDLQARFGGYWGQGTQVTVSSEPKTPKPTSGR

SEQ ID NO: 41 - protein, 131 aa, synthetic construct
QVKLQQSGGGLVQPGGSLRLSCAASESTPSINTMGWYRQAPGKERELVATITSGGMTNYADSVKGRFTISRDNGKNTVYLQMNSLEPGDTAVYYCNLKRRDLQARFGGYWGQGTQVTVSSEPKTPKPQSGR

SEQ ID NO: 42 - protein, 132 aa, synthetic construct
QVKLQQSGGGLVQPGGSLRLSCAASGFTFRNYAMSWVRQAPGKGLEWVSAIAAGGAVTKYADSVKGRFTISRDNARDTLYLQMNSLKPEDTAVYYCAKPRDFWYSPEFDFRGQGTQVTVSSEPKNQNQTSGR

SEQ ID NO: 43 - protein, 133 aa, synthetic construct
QVQLQQSGGGLVQAGDSLTISCASSLFTFSTSTMGWFRQAPGKEREFVAAIKSSGSSMYYADSVQGRFTISRDNAKKTVTLQMNSLKPEDTAVYYCAKGVYGSRRSADFGSWGQGTQVTVSSEPKTPKPQSGR

SEQ ID NO: 44 - protein, 133 aa, synthetic construct
QVQLQQSGGGLVQAGDSLRISCAASLFTFSTSTMGWFRQAPGKEREFVAAIRSTGDSMYYADSVQGRFTISRDNAKKMVYLQMNNLKPEDTAVYYCAKGVYGSRRSADFGSWGQGTQVTVSSEPKTPKPQSGR

SEQ ID NO: 45 - protein, 133 aa, synthetic construct
DVQLQASGGGLVQAGGSLRISCTADGYTFSTSTMGWFRQAPGKEREFVAAIKSDGSIMYYADSVAGRFIISRDNAKKMVFLQMDRLKPEDTAVYYCAKGVYGSRRSADFGWWGQGTQVTVSSEPKTPKPQSGR

SEQ ID NO: 46 - protein, 134 aa, synthetic construct
QVKLQESGGGLVQAGESLRLSCAASGSNFSINGVGWYRQAPGKQRELVAGITNGGYTSYADSVKGRFTISTDNAKNTVYLQMNSLRPEDTAVYYCNANFQIHRSGADYVRNYWGQGTQVTVSAEPRTKTTTSGR

SEQ ID NO: 47 - protein, 134 aa, synthetic construct
QVKLQQSGGGLVQAGGSLRLSCAASGNFFTLNGVAWYRQAPGKQRELVAGITSGGWTNYADSVKGRFTISADNAKNTVYLQMNSLRPEDTAVYYCNANLQIHRDSSGDVRNVWGQGTQVTVSSEPKTPKPQSGR

SEQ ID NO: 48 - protein, 136 aa, synthetic construct
DVQLQASGGGLVQAGGSLRLSCAASGSFFSINGVGWYRQAPGKQRELVAGITNGGFTNYADSVKGRFTISRDNAKNTVYLQMNSLKPEDTAVYYCNANLQISRSEDGAYVVRNYWGQGTQITVSSEPKTPKPQSGR

SEQ ID NO: 49 - protein, 134 aa, synthetic construct
DVQLQASGGGLVQAGGSLRLSCAASGSGFSINGVGWYRQTPGRQRELVAGITIGGYTNYADSVKGRFTISSDNAKNTVYLQMNSLKPEDTAVYYCNANLQFYRGGGSDVKNYWGQGTQVTVSSEPKTPKPQSGR

SEQ ID NO: 50 - protein, 135 aa, synthetic construct
QVQLQESGGGLVQAGGSLRLSCAASGLTFSSYAMGWFHQAPGKEREFVAAINWSGRDTYYADSVKGRFTISRDNRKNTVYLQMNSLKPEDTAVYYCAAAEFFSSGDPLPGMDYWGKGTLVTVSSEPKTQNHNSGR

SEQ ID NO: 51 - protein, 135 aa, synthetic construct
QVKLQQSGGGLVQAGGSLRLSCAASGLTFSSYAMGWFRQAPGKEREFVAAINWSGRDTYYADSVKGRFTISRDNAKNTVYLQTNSLKPEDTAVYYCAAAEFLPTQRSPREYDYWGLGTQVTVSSEPKTPKPQSGR

SEQ ID NO: 52 - protein, 136 aa, synthetic construct
QVKLQQSGGGLVQAGGSLRLSCAASGYAFNTYTMAWFRQAPGKEREFVALVGMKVDGKIYADSVKGRFTISRDNEQKTVLLEMNHLEPEDTAIYYCAASRRFWTAALNGADYPYWGQGTQVTVSSEPKTPKPQSGR

SEQ ID NO: 53 - protein, 129 aa, synthetic construct
QVKLQQSGGGLVQAGGSLRLSCAASGIVFSFNAMGWYRVPPGKQRELVADILKSGGTNVVDSVKGRFAISRDSAQNTLYLQMNRLKPEDTAVYYCNARDWSDGFDEYWGQGTQVTVSSEPKTPKPQSGR

SEQ ID NO: 54 - protein, 135 aa, synthetic construct
QVQLQQSGGGLVQAGGSLRLSCSASGRTFSNYVMGWFRQAPGKEREFVATISASGGSTYCADSVEGRFTISRDNAKNTAYLQMNNLEPEDTAVYYCASGPRANASIRRSGYNYWGQGTQVTVSSEPKTPKPQSGR

SEQ ID NO: 55 - protein, 136 aa, synthetic construct
QVQLQQSGGGLVQAGGSLRLSCAASGLAFSSYAITWLRQAPGTEREFVALISGSGSSTYYADSVKGRFTISRNNAKNTVYLQMNSLKPEDTAVYYCTASEFLLHPPPPNQKYDYWGQGTQVTVSSEPKTPKHTSGR

SEQ ID NO: 56 - protein, 135 aa, synthetic construct
QVQLQQSGGGLVRAGGSLRLSCSASGRTFGNEVMGWFRQAPGKEREFVAAINWSSGNTYYRDSVKGRFTISRDNAKNTVYLQMNSLEPEDTAVYYCAARSRPAISTRRPDYFAWGQGTQVTVSSEPKTPKPQSGR

SEQ ID NO: 57 - protein, 133 aa, synthetic construct
QVQLQQSGGGLVQAGGSLRLSCTASGRIDRTYTVSWFRQGPGKEREFVATISWDGSIYYDNAVEGRFSISGDNAKTTVALQMNSLKPEDTAVYYCAARRRVFSRAAAAYNYWGQGTQVTVSSEPKTPKPQSGR

SEQ ID NO: 58 - protein, 124 aa, synthetic construct
QVKLQQSGGGLVQPGGSLRLTCAASGFPFSTYAIRWVRRPPGKGLEWVSTIHPDFTTNYADSVSRRFTISRDNAKNTVYLQMNSLKPEDTAVYYCSRGVSGERGQGTQVTVSSEPKTPKPHSGR

SEQ ID NO: 59 - protein, 132 aa, synthetic construct
QVQLQQSGGGLVQPGGSLRLSCAASGSIFSIHTMGWYRQAPGKQRELVTTITTGGTTNYADSVKGRFTISRDDAKNTVYLQMNNLKPEDTAVYYCYALIQTASTTWYRQYWGQGTQVTVSSEPKTQNHNSGR

SEQ ID NO: 60 - protein, 126 aa, synthetic construct
QVKLQESGGGLVQPGGSLRLSCTASRSIFTRAMAWYRQAPGKQRELVAAIDSGDRTHYADSVKGRFTISRNNAKDTLYLQMNSLKSEDTAVYYCNANLGALLDYWGQGTQVTVSSEPKTQTTTSGR

SEQ ID NO: 61 - protein, 129 aa, synthetic construct
QVKLQESGGGLVQAGGSLRLSCAASGFTFATYAMAWYRQAPGKQRELVASISNFGSTAYGDSVKGRFTISRDNAKNTVYLEMNSLKSEDTAVYYCKRVRDVIGRPELWGQGTQVTVSSEPKTPKPHSGR

SEQ ID NO: 62 - protein, 132 aa, synthetic construct
QVKLQESGGGLVQPGGSLRLSCLPSINIFSLAAMGWYRQAPGKQRELVASISSGGTANYADSYADSVKGRFTISRDIAKNTVDLQMNSLKPEDTAVYYCKVDSYTYGTDIWGKGVLVTVSSEPQDTKTTSGR

SEQ ID NO: 63 - protein, 140 aa, synthetic construct
QVKLQQSGGGLVQAGGSLRLSCAASGTFSRYAMGWFRQAPGKEREFVAAINWTGGSTYYADSVKGRFTISGDNAKNTVYLQMNSLKPGDTAVYYCAAEVHPGDYGLTYMQSQYEYDYWGQGTQVTVSSEPKTPKPTTSGR

SEQ ID NO: 64 - protein, 131 aa, synthetic construct
DVQLQASGGGLVQAGGSLRLSCAASTSRFSSYAMGWSRQAPGKQRELVASISSSGLTTNYADSVKGRFTISRDNAKNTVYLQMNSLKPEDTAVYYCKADGRRYSLNEYWGQGTQVTVSSEPKTPKPQPSGR

SEQ ID NO: 65 - protein, 133 aa, synthetic construct
DVQLQASGGGLVQAGGSLRLSCAASGSTISSYAMAWYRQAPGKRRELVAHISSGGSTNYADSVKGRFTISRDNAKNTVYLQMNSLKPEDTAVYYCNIYYGGDYYYTGVKPNPWGQGTQVTVSSAHHSEDPRGR

SEQ ID NO: 66 - protein, 137 aa, synthetic construct
QVKLQESGGGWVQAGGSLRLSCAASALTASITTMGWFRQTPEKEREFLAAINWTGDYKYYADSVKGRFTISRDNAKNTVDLQMNQLKPEDTAVYYCAASKIRNDIYLNDYTWYQYWGQGTQVTVSSEPKTPKPQSGR

SEQ ID NO: 67 - protein, 143 aa, synthetic construct
DVQLQASGGGLVQAGGSLRLSCVASARTFSSYAMGWFRQAPGKEREFVAAISWSGASTDYADSVKGRFTISRDNAKKTVYLQMNTLKPEDTAVYYCAAHHITPTGSYYYSEPLPVDMVYDYWGQGTQVTVSSEPKTKTTTSGR

SEQ ID NO: 68 - protein, 8 aa, synthetic construct
AEAAAKAS

SEQ ID NO: 69 - protein, 13 aa, synthetic construct
AEAAAKEAAAKAS

SEQ ID NO: 70 - protein, 23 aa, synthetic construct
AEAAAKEAAAKEAAAKEAAAKAS

SEQ ID NO: 71 - protein, 12 aa, synthetic construct
APAPSPAPSPAS

SEQ ID NO: 72 - protein, 23 aa, synthetic construct
TVIIEYAHRISQDILSDAIQQWA

SEQ ID NO: 73 - protein, 24 aa, synthetic construct
TVIIEYAHEISQDILSDAIQQSAG

SEQ ID NO: 74 - protein, 23 aa, synthetic construct
TVIIEYAHELSQDILSDALQQWA

SEQ ID NO: 75 - DNA, 6269 bp, synthetic construct
  source 1..6269: mol_type = other DNA
cagcgatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggcctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatactatgggaaaaatagaggaagggaaattagtcatctggatcaacggcgataagggatacaacggcctggccgaggttggcaaaaaatttgaaaaagataccggaataaaagttacggtggaacatccagataagttggaagaaaaattccctcaagtcgctgccaccggcgacggtccagatataatattttgggcacatgatcggtttgggggttatgcccagtccggactgctggcggaaataacgccggacaaagcttttcaagataagctctatccgttcacctgggatgcagttcgctataatgggaaactgattgcatacccgatcgccgtggaagctctgagccttatttataacaaggacttactgccgaatccgcctaaaacgtgggaggaaattccggctctggataaggaactgaaagccaaaggcaagtcagcattaatgtttaaccttcaggaaccttattttacctggccactgattgcagccgacggggggtatgcgtttaaatacgagaacggtaagtatgatataaaggatgtgggtgttgataatgcaggggcgaaagccggcttgactttcttggtggacctgatcaaaaacaaacacatgaacgcagatacggattactcgattgctgaggcagcatttaataaaggcgagacggcaatgacaataaacggcccatgggcatggagcaatattgacacttccaaggtcaattatggagttaccgttctgccgacatttaaaggccaaccgtccaagccattcgttggtgttctgtcggccggtataaatgccgcttctccgaataaagaacttgcgaaggaatttctcgaaaattatctcttgaccgatgagggtctcgaggccgtcaataaagataaaccgctgggtgcggtagccctcaaaagctatgaagaagagctggtcaaagatcctcgcatagccgctacgatggaaaatgcccagaaaggggaaataatgccgaacattcctcagatgagcgcattctggtatgccgtacgaacggcagttataaacgccgcgagcggccgtcaaacagttgatgaagctctgaaagatgcccaaaccacgggttctggatccacagttataattgagtatgcccacgaactgagtcaagatatactcagcgatgcattgcaacagtgggccggatcaccggcaccggctcccgcgggaaatccgaacgccaatcccaatgcaaatccgaacgccaatccaaatgcaaatccaaacgccaaccccaatgccaatccgaacgccaacccggggggtagttggtcgcaccctcagtttgaaaaaggtggatctcaccatcaccaccatcattaataataactcgagcaccaccaccaccaccactgagatccggctgctaa

SEQ ID NO: 76 - protein, 455 aa, synthetic construct
MGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTTGSGSTVIIEYAHELSQDILSDALQQWAGSPAPAPAGNPNANPNANPNANPNANPNANPNANPNANPGGSWSHPQFEKGGSHHHHHH